(19) United States
(12) Patent Application Publication
(10) Pub. No.: US 2021/0340232 A1
(43) Pub. Date: Nov. 4, 2021

(54) MONOCLONAL ANTIBODIES AGAINST HUMAN DICKKOPF3 AND USES THEREOF

(71) Applicant: Board of Regents, The University of Texas System, Austin, TX (US)

(72) Inventors: Rosa HWANG, Houston, TX (US); Liran ZHOU, Houston, TX (US); Mason LU, Houston, TX (US); Hongmei HUSTER, Houston, TX (US); Craig LOGSDON, Houston, TX (US); Jeffrey E. LEE, Houston, TX (US)

(73) Assignee: Board of Regents, The University of Texas System, Austin, TX (US)

(21) Appl. No.: 17/285,470

(22) PCT Filed: Oct. 15, 2019
(86) PCT No.: PCT/US2019/056355

§ 371 Date: Apr. 14, 2021

Related U.S. Application Data
(60) Provisional application No. 62/745,671, filed on Oct. 15, 2018.

(51) Int. Cl. C07K 16/18; A61K 47/68; A61P 35/00; A61K 45/06; A61K 39/395; A61K 31/7068

(52) U.S. Cl. CPC C07K 16/18; A61K 47/6843; A61P 35/00; A61K 2039/505; A61K 39/3955; A61K 31/7068; A61K 45/06

(57) ABSTRACT

Provided herein are methods and reagents for increasing chemosensitivity to chemotherapy and immunotherapy in cancer patients. Methods of treating cancer are provided, comprising administering to a patient in need thereof an effective amount of an DKK3-neutralizing agent, such as a DKK3-neutralizing antibody provided herein. The methods can further include administering an effective amount of chemotherapy or immunotherapy to said patient.

### REFERENCE TO RELATED APPLICATIONS

The present application claims the priority benefit of U.S. provisional application No. 62/745,671, filed Oct. 15, 2018, the entire contents of which is incorporated herein by reference.

### STATEMENT REGARDING FEDERALLY SPONSORED RESEARCH

This invention was made with government support under grant numbers CA016672 and CA218495 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Oct. 1, 2019, is named UTFCP1395WO_ST25.txt and is 9.5 kilobytes in size.

### BACKGROUND

### 1. Field

The present invention relates generally to the fields of medicine, immunology, and cancer biology. More particularly, it concerns antibodies that neutralize Dickkopf3 (DKK3) and methods of their use.

### 2. Description of Related Art

The tumor microenvironment is recognized as an important mediator of tumor progression for many cancers (Hwang et al., 2008; Kalluri & Zeisberg, 2006; Apte et al., 2004; Apte & Wilson, 2012; Bhowmick & Moses; 2005; Dvorak, 1986), and pancreatic ductal adenocarcinoma (PDAC) in particular is characterized by a dense fibrotic stroma in the tumor microenvironment. This fibrotic stroma consists primarily of pancreatic stellate cells (PSCs), which promote PDAC proliferation and metastasis (Apte & Wilson, 2012; Hwang et al., 2008; Xu et al., 2010) and reduce PDAC cell responses to therapeutics (Hwang et al., 2008; Olive et al., 2009). However, the precise mechanisms of how PSCs affect these processes are not well understood and as a result, clinical trials targeting the stroma in PDAC have had largely disappointing results (Bijlsma & van Laarhoven, 2015). Previous efforts to target PDAC stroma were directed at broadly eliminating stromal elements including fibroblasts. More effective strategies to inhibit specific tumor-promoting mechanisms elaborated by PSCs are needed.

### SUMMARY

In one embodiment, monoclonal antibodies or antibody fragments are provided, where the antibodies or antibody fragments are characterized by clone-paired heavy and light chain CDR sequences from Tables 1 and 2, respectively. In one aspect, the antibody or antibody fragment has light chain variable sequence CDRs 1-3 according to SEQ ID NOs: 1, 2, and 3, respectively, and heavy chain variable sequence CDRs 1-3 according to SEQ ID NOs: 7, 8, and 9, respectively. In one aspect, the antibody or antibody fragment has light chain variable sequence CDRs 1-3 according to SEQ ID NOs: 4, 5, and 6, respectively, and heavy chain variable sequence CDRs 1-3 according to SEQ ID NOs: 10, 11, and 12, respectively. In various aspects, any given CDR sequence may vary from those of Tables 1 and 2 by one or two amino acid substitutions. In various aspects, any given CDR sequence may have an at least 70%, 75%, 80%, 85%, 90%, or 95% identity to those of Tables 1 and 2.

In some aspects, the antibodies or antibody fragments are encoded by light and heavy chain variable sequences according to clone-paired sequences from Table 3. In one aspect, the antibody or antibody fragment has a heavy chain variable sequence encoded by a nucleic acid sequence according to SEQ ID NO: 13 and a light chain variable sequence encoded by a nucleic acid sequence according to SEQ ID NO: 14. In one aspect, the antibody or antibody fragment has a heavy chain variable sequence encoded by a nucleic acid sequence according to SEQ ID NO: 15 and a light chain variable sequence encoded by a nucleic acid sequence according to SEQ ID NO: 16. In one aspect, the antibody or antibody fragment has a heavy chain variable sequence encoded by a nucleic acid sequence having at least 70%, 80%, 90%, or 95% identity to SEQ ID NO: 13 and a light chain variable sequence encoded by a nucleic acid sequence having at least 70%, 80%, 90%, or 95% identity to SEQ ID NO: 14. In one aspect, the antibody or antibody fragment has a heavy chain variable sequence encoded by a nucleic acid sequence having at least 70%, 80%, 90%, or 95% identity to SEQ ID NO: 15 and a light chain variable sequence encoded by a nucleic acid sequence having at least 70%, 80%, 90%, or 95% identity to SEQ ID NO: 16.

In some aspects, the antibodies or antibody fragments comprise light and heavy chain variable sequences according to clone-paired sequences from Table 4. In one aspect, the antibody or antibody fragment comprises a heavy chain variable sequence according to SEQ ID NO: 17 and a light chain variable sequence according to SEQ ID NO: 18. In one aspect, the antibody or antibody fragment comprises a heavy chain variable sequence according to SEQ ID NO: 19 and a light chain variable sequence according to SEQ ID NO: 20. In one aspect, the antibody or antibody fragment comprises a heavy chain variable sequence having at least 70%, 80%, 90%, or 95% identity to SEQ ID NO: 17 and a light chain variable sequence having at least 70%, 80%, 90%, or 95% identity to SEQ ID NO: 18. In one aspect, the antibody or antibody fragment comprises a heavy chain variable sequence having at least 70%, 80%, 90%, or 95% identity to SEQ ID NO: 19 and a light chain variable sequence having at least 70%, 80%, 90%, or 95% identity to SEQ ID NO: 20.

Also provided herein are monoclonal antibodies or antigen binding fragments thereof, which compete for binding to the same epitope as any of the monoclonal antibodies or an antigen-binding fragments thereof that are defined herein based on their CDR sequences.

In some aspects, the antibody or antibody fragments are humanized antibodies. In some aspects, the antibody fragments are a monovalent scFv (single chain fragment variable) antibodies, divalent scFv, Fab fragments, F(ab′)₂ fragments, F(ab′)₃ fragments, Fv fragments, or single chain antibodies. In some aspects, the antibodies are chimeric antibodies or bispecific antibodies. In some aspects, the antibodies are IgG antibodies or recombinant IgG antibodies or antibody fragments. In some aspects, the antibodies or antibody fragments are conjugated or fused to an imaging agent or a cytotoxic agent.

In one embodiment, hybridomas or engineered cells encoding antibodies or antibody fragments of the present embodiments are provided.

In one embodiment, methods of treating a patient having cancer, the methods comprising administering an effective amount of a DKK3-neutralizing antibody or antibody fragment. In some aspects, the DKK3-neutralizing antibody or antibody fragment is an antibody or antibody fragment of the present embodiments. In some aspects, the cancer patient has been determined to express an elevated level of DKK3 relative to a control patient. In some aspects, the cancer patient has been determined to express a decreased level of DKK3 relative to a control patient. In some aspects, the cancer patient has been determined to express an altered or abnormal level of DKK3 relative to a control patient. In some aspects, the cancer patient has been determined to express a normal level of DKK3 relative to a control patient.

In some aspects, the methods are further defined as methods for increasing sensitivity to chemotherapy. In some aspects, the methods are further defined as methods for increasing sensitivity to immunotherapy. In some aspects, the cancer is a pancreatic cancer, breast cancer, ovarian cancer, gastric cancer, bladder cancer, or sarcoma. The breast cancer may be triple-negative breast cancer. In some aspects, the methods are further defined as methods of inhibiting cancer metastasis. Also or alternatively in some aspects, the methods are further defined as methods of inhibiting cancer growth.

In some aspects, the methods further comprise administering at least a second anti-cancer therapy. In certain aspects, the second anti-cancer therapy is a chemotherapy, immunotherapy, radiotherapy, gene therapy, surgery, hormonal therapy, anti-angiogenic therapy or cytokine therapy. In one aspect, the chemotherapy comprises gemcitabine. In certain aspects, the immunotherapy comprises an immune checkpoint inhibitor. In certain aspects, the immune checkpoint inhibitor is a CTLA-4 antagonist, a PD-1 antagonist, a PD-L1 antagonist, an OX40 agonist, a LAGS antagonist, a 4-1BB agonist, or a TIM3 antagonist. In certain aspects, the immune checkpoint inhibitor is a combination of a CTLA-4 antagonist and a PD1 antagonist. In certain aspects, the immune checkpoint inhibitor is a combination of a CTLA-4 antagonist and a PDL1 antagonist.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-G. DKK3 is expressed by HPSCs in PDAC. DKK3 expression was measured in HPSCs and PDAC cell lines by RT-PCR (FIG. 1A) and qPCR (FIG. 1B) in mono- and co-culture. Striped bars indicate expression in HPSCs after co-culture with PDAC cells. (FIG. 1C) DKK3 expression in human PDAC and normal pancreatic tissue was determined by Affymetrix array. (FIG. 1D) DKK3 levels were measured by ELISA in plasma samples from patients with PDAC, chronic pancreatitis (CP), or no pancreatic disease and in conditioned media from HPSCs (HPSC-CM). (FIG. 1E) IHC analysis of DKK3 in a tissue microarray of human PDAC. Shown are representative fields at 100× magnification; inset magnification is 200×. (FIG. 1F) In a genetically engineered mouse model (GEMM) of PDAC, DKK3 is expressed early in development with CP and pancreatic intraepithelial neoplasia (PanIN) lesions and progresses in PDAC. (FIG. 1G) Relative expression in the GEMM of PDAC and in cancer cells isolated from GEMM tumors was quantified by Affymetrix. *p<0.05, ***p<0.001. Values are mean±SEM.

FIGS. 2A-J. DKK3 stimulates HPSC and PDAC activity and increases chemoresistance. (FIG. 2A) HPSC proliferation was measured by MTT after treatment with PBS or rhDKK3 (10 μg/ml). DKK3 was silenced in HPSCs by shDKK3 (FIG. 2B) and cell proliferation was measured by MTT assay (FIG. 2B) and migration was determined at 24 hours (FIG. 2C). Control cells were transfected with scrambled shRNA. (FIG. 2D) Panc1 cells were treated with rhDKK3 (10 μg/ml) or serum-free media control, and cell migration and invasion were measured after 24 hours. (FIGS. 2E-F) Panc1 cells were stably silenced for DKK3 and cell proliferation (FIG. 2E) and colony formation in soft agar (FIG. 2F) were measured. (FIG. 2G) BxPC3 cell migration was measured after treatment with CM from HPSCs or HPSCs silenced for DKK3. Soft agar colony formation (FIG. 2H) in gemcitabine and apoptosis (FIG. 2I) was determined in chemosensitive L3.6p1 cells expressing DKK3 compared with transfection controls. (FIG. 2J) Gemcitabine-induced apoptosis was measured in chemoresistant HS766T cells silenced for DKK3. *p<0.01, **p<0.001, ***p<0.0001, ****p<0.00001.

FIGS. 3A-F. NF-κB is activated in PSCs and PDAC cells by DKK3 and is necessary for DKK3-mediated stimulation of cell activity. (FIG. 3A) Phosphorylation of p65 and IκBα induced by DKK3 treatment (10 μg/ml) was determined by Western blotting. Relative protein loading was shown by using anti-β-actin antibody. (FIG. 3B) Time course of p65 activation by WB in HPSC and Panc1 cells. Cells were treated with recombinant DKK3 (10 μg/ml) for 0-24 h and change in band density relative to baseline were quantified. (FIG. 3C) DKK3 stimulates NFκB luciferase reporter in HPSC & PDAC cells, with mutant luc reporter (MT). NFκB activity induced by DKK3 was measured in Panc28 with phosphorylation-defective IκBαM by luciferase reporter (FIG. 3D) and Western blotting (FIG. 3E). For FIGS. 3C&D, in each set of three columns, the left is “PBS,” the middle is “DKK3,” and the right is “TNFα.” (FIG. 3F) Proliferation of Panc28 and Panc28/IκBαM was measured by MTT assay. *p<0.05, **p<0.01, ***p<0.001 and ****p<0.0001 vs. PBS control.

FIGS. 4A-H. Neutralization of DKK3 inhibits tumor growth and prolongs survival. BxPC3 tumor cells labeled with firefly luciferase were orthotopically implanted into nude mice, with or without either control HPSCs or HPSCs stably silenced for DKK3, in a 1:3 tumor:stroma ratio. (FIG. 4A) Average pancreas tumor volume at 35 days post-injection. (FIG. 4B) Tumor growth by IVIS imaging of Panc02 tumor cells implanted subcutaneously in syngeneic C57/BL6 or DKK3-null mice with Ki67 expression by IHC. DKK3-deficient mice were crossed with KPC mice to produce P48-Cre; Kras LSL-G12D;Trp53fl/fl; dkk3−/− progeny. (FIGS. 4C-D) Kaplan Meier survival curve and survival table for mice with wild-type DKK3 (left line), DKK3-null (right-most line), or heterozygous DKK3 (middle line). (FIG. 4E) Representative images of tumors from (FIG. 4C) from DKK3-wild type mice (moribund, day 47) or DKK3-heterozygous mice (moribund, day 63) or DKK3-null mice (early time point at day 48, or when moribund at day 68). (FIGS. 4F-H) DKK3 and collagen type I expression by qPCR and Ki67 proliferation index by IHC are shown. * p<0.05.

FIGS. 5A-H. DKK3-blocking antibodies inhibit PSC and cancer cell activity, chemoresistance and tumor progression with improved survival. HPSCs and BxPC3 cells were treated with DKK3 mAb clones JM6-6-1 and JM8-12-1 or isotype control mAb or PBS. (FIGS. 5A-B) HPSC apoptosis and migration as measured by FACS and Transwell migration assay at 48 hours. (FIG. 5C) BxPC3 migration in response to rhDKK3 10 μg/ml as measured by Transwell migration assay at 48 hours. (FIG. 5D) BxPC3 resistance to gemcitabine 100 μM as measured by MTS proliferation assay at 6 days. HPSC-CM, pancreatic stellate cell conditioned media, 10 μg/ml. The orthotopic co-injection BxPC3+ HPSC model of PDAC was used to test the efficacy of DKK3 mAb clones JM6-6-1 or JM8-12-1 (5 mg/kg i.p. once every 5 days). (FIG. 5E) Overall tumor progression was measured every 3-4 days by IVIS imaging. (FIG. 5F) Metastatic tumors in the peritoneal cavity after removal of the primary pancreatic tumor are shown by IVIS imaging. (FIG. 5G) Kaplan Meier survival curve showing mice treated with DKK3 mAb clone JM8-12 (right-most line), control mAb (middle line), or PBS (left-most line). (FIG. 5H) KPC mice (P48-Cre; Kras LSL-G12D;Trp53fl/fl) with either wild type DKK3 (solid lines) or deficient in DKK3 (“DKK3-KO,” dashed lines) were treated with DKK3 mAb JM6-6-1 (5 mg/kg i.p. once every 5 days), PBS or control mAb. Kaplan Meier survival curve is shown with hazard ratios (Log-rank test). *p<0.05, **p<0.01, ***p<0.001, ****p<0.0001 vs. control Ab.

FIGS. 6A-F. DKK3 blockade is associated with increased tumor immune infiltrates and improves response to checkpoint inhibitor therapy. (FIG. 6A) T cells were stimulated, treated with DKK3 (5-10 μg/mL) and proliferation was measured by CFSE assay. In a syngeneic orthotopic model with luciferase-labeled KPC cells, tumors were examined for CD3 and CD8 expression by IHC (FIG. 6B) and additional markers of T cell activity were measured by quantitative PCR (FIG. 6C). Mice in this model were treated with either control IgG, DKK3 mAb JM6-6-1, αCTLA4 or the combination JM6-6-1+αCTLA4 and tumor growth was measured by IVIS imaging to 25 and 190 days (FIG. 6D). Survival in this orthotopic implantation model is shown in (FIG. 6E). Using a GEMM (FIG. 6F), KPC/DKK3^{+/+} (black line) or KPC/DKK3^{-/-} (blue line) mice were treated with αCTLA4 or control IgG and the Kaplan Meier survival curve is shown. *p<0.05, **p<0.01, ***p<0.001.

FIGS. 7A-E. DKK3 expression and silencing. (FIG. 7A). DKK3 protein was detected by Western blotting in HPSCs and HPSC conditioned media (CM). (FIG. 7B). DKK3 was silenced in HPSC, Panc1 and HS766T cells by shRNA or siRNA. (FIG. 7C) DKK3 expression by RT-PCR in HPSCs derived from four patients. (FIG. 7D) DKK3 expression by RT-PCR in HUVEC and HPSCs. (FIG. 7E) DKK3 and αSMA expression in human PDAC by IHC.

FIGS. 8A-H. DKK3 expression and function in HPSC and PDAC cells. Primary HPSC cells 20A were derived from patient with PDAC and expressed DKK3 at a similar level to HPSC by RT-PCR (FIG. 8A). Silencing of DKK3 by shRNA (FIG. 8B) resulted in inhibition of cell proliferation by MTS assay and cell migration (FIGS. 8C-D). (FIG. 8E) BxPC3 cells were treated with rhDKK3 (10 μg/ml) and cell migration and invasion were measured after 24 hours. (FIGS. 8F-G) Dose response curves for DKK3 effects on HPSC proliferation and BxPC3 migration. (FIG. 8H) Western blot for DKK3 in rhDKK3 and HPSC-CM. ***p<0.0001, ****p<0.0001

FIG. 9. DKK3 expression in syngeneic models of PDAC. DKK3 expression was determined by RT-PCR and qPCR in mouse PSCs, Panc02 cells and tumors from subcutaneously injected Panc02 cells in DKK3^{-/-} mice (DKK3-KO) or control C57/BL6 wild-type (WT) mice (DKK3^{+/+}). MPSC=mouse PSCs; NP=normal pancreas.

FIG. 10. Depletion of DKK3 is associated with improved survival in the P48-Cre; Kras^{LSL-G12D};Trp53^{fl/+} model of PDAC. Kaplan-Meier survival curves are shown (log rank test). The left line is DKK3 wt/p53+/−; the middle line is DKK3−/−/p53+/−; the right line is DKK3+/−/p53+/−.

FIGS. 11A-C. DKK3 expression in autochthonous model of PDAC and effects of DKK3 mAb on cell surface binding of DKK3 and orthotopic model. (FIG. 11A) DKK3 expression was measured by qPCR in pancreatic tumors from P48-Cre; Kras LSL-G12D;Trp53fl/fl and P48-Cre; Kras LSL-G12D;Trp53fl/+ mice that are either wild type DKK3 or heterozygous or homozygous DKK3-null. Relative expression of DKK3 mRNA in DKK3+/− hetero mice is 53% of DKK3^{+/+} mice. (FIG. 11B) DKK3 cell surface binding was assessed by incubating BxPC3 or L3.6p1 cells with His-tagged rhDKK3 (10 μg/ml) with or without JM6-6-1 (70 μg/ml) and positive cells were sorted by flow cytometry. (FIG. 11C) Mice bearing orthotopic BxPC3 tumors were treated with DKK3 mAb JM6-6-1 (5 mg/kg i.p. once every 5 days). Overall tumor progression was measured every 3-4 days by IVIS imaging.

FIGS. 12A-D. DKK3 expression in mouse PSCs and effects of treatment with DKKC mAb on survival. (FIG. 12A) Western blot analysis of recombinant human and mouse DKK3 under denaturing and non-denaturing conditions using JM6-6-1 mAb. (FIG. 12B) Expression of murine DKK3 in murine PSCs (MPSC) or 3T3 cells was determined by RT-PCR. 18S was used as loading control. (FIG. 12C) Proliferation of MPSCs treated with DKK3 mAb was measured at 7 days by MTS assay. ***p<0.001 vs. PBS. (FIG. 12D) Pdx1-Cre; Kras LSL-G12D;Trp53fl/+ model of PDAC was treated with DKK3 mAb JM6-6-1 or control mAb JM4-74 (5 mg/kg i.p. q5d). Kaplan-Meier survival curves are shown (log rank test). At 0% survival, the right line is JM6-6-1, the middle lines is PBS, and the left line is Control Ab.

FIGS. 13A-F. DKK3 expression in triple negative breast cancer (TNBC) and association with clinical outcome and cell proliferation. (FIG. 13A) DKK3 expression was measured by RT-PCR in TNBC fibroblasts (BCF), human pancreatic stellate cells (HPSC) from pancreatic adenocarcinoma (PDAC), BxPC (PDAC cancer cell) and water control. (FIG. 13B) DKK3 protein was measured by Western blotting in BCF from TNBC, in ER-positive breast cancer cells, and in TNBC cancer cell lines. (FIG. 13C) DKK3 protein was measured in patient-derived xenograft (PDX) tumors from TNBC relative to recombinant human DKK3 (rhDKK3) and conditioned medium from HPSC (HPSC-CM). Actin was used as loading control. (FIG. 13D) Correlation between DKK3 expression in TNBC human tumors and patient survival is shown in Kaplan Meier plot. Data is pooled from TCGA, EGA and GEO. The top line is “low” and the bottom line is “high.” (FIG. 13E) SUM159 TNBC cell proliferation is shown with rhDKK3 treatment. The top line is “20 μg/ml DKK3,” the middle line is “5 μg/ml DKK3,” and the bottom two lines are “Vehicle” and “2.5 μg/ml DKK3.” (FIG. 13F) The 4T1 mouse model of TNBC was treated with JM6-6-1, and the Kaplan Meier plot shows that the treatment increased survival relative to the control antibody 4-74 and PBS. The left line is PBS; the middle line is Control Ab 4-74; the right line is JM6-6-1.

FIGS. 14A-C. Treatment of TNBC orthotopic model with DKK3 mAb. (FIG. 14A) Orthotopic model using 4T1 TNBC cells (labeled with firefly luciferase) was treated with PBS, control IgG Ab or anti-DKK3 mAb JM6-6-1 (5 mg/kg ip q5 days). Tumor growth was measured by calipers at day 33 after starting treatment and compared to initial tumor size prior to treatment. (FIG. 14B) 4T1 primary tumors and metastases were imaged using IVIS at day 33 after starting treatment. (FIG. 14C) Luciferase signal from 4T1 metastases was measured by IVIS at day 33 after starting treatment.

FIGS. 15A-E. DKK3 expression in various cancer types is associated with clinical outcome. (FIG. 15A) Correlation between DKK3 expression in human ovarian cancers and patient survival is shown in Kaplan Meier plot. (FIG. 15B) Correlation between DKK3 expression in human gastric cancers and patient survival is shown in Kaplan Meier plot. (FIG. 15C) Correlation between DKK3 expression in human PDAC and patient survival is shown in Kaplan Meier plot. (FIG. 15D) Correlation between DKK3 expression in human bladder cancers and patient survival is shown in Kaplan Meier plot. (FIG. 15E) Correlation between DKK3 expression in human sarcomas and patient survival is shown in Kaplan Meier plot.

### DETAILED DESCRIPTION

Pancreatic ductal adenocarcinoma (PDAC) has a dismal prognosis and whether its stromal infiltrate contributes to its aggressiveness is unclear. Here, Dickkopf-3 (DKK3) was found to be produced by pancreatic stellate cells and present in the majority of human PDAC. DKK3 stimulates PDAC growth, metastasis, and resistance to chemotherapy with both paracrine and autocrine mechanisms through NF-κB activation. Genetic ablation of DKK3 in an autochthonous model of PDAC inhibited tumor growth, induced a peritumoral infiltration of CD8+ T cells, and more than doubled survival. Treatment with a novel DKK3 blocking monoclonal antibody inhibited PDAC progression and chemoresistance and prolonged survival. The combination of DKK3 inhibition with checkpoint control inhibition was more effective in reducing tumor growth than either treatment alone and resulted in a durable improvement in survival, suggesting that DKK3 neutralization is effective as a single targeted agent or in combination with chemo- or immuno-therapy for cancer.

### I. Dickkopf-3 (DKK3)

DKK3 is a 38-kDa member of the dickkopf (Dkk) family of glycoproteins (DKK1-4) that may be involved in regulating Wnt pathways (Macheda & Stacker, 2008; Moon et al., 2004; Taipale & Beachy, 2001). The best-characterized member of the DKK family is DKK1, which is a natural soluble inhibitor of Wnt signaling and is associated with tumor suppressor functions (Cowling et al., 2007; Shou et al., 2002). DKK3 shares a unique N-terminal cysteine-rich domain and C-terminal colipase fold domain with other Dkks but otherwise, Dkk3 appears to be a divergent member of the Dkk family with differences in DNA sequence, chromosome group location, and potentially receptor and signaling mechanisms as well (Guder et al., 2006; Niehrs, 2006).

In contrast to DKK1, the functional role of DKK3 in cancer is not clear with conflicting reports of its effect as either a tumor suppressor or promoter. In prostate cancer and osteosarcoma, DKK3 is described as a tumor suppressor and its overexpression inhibits tumor growth and metastasis (Abarzua et al., 2005; Edamura et al., 2007; Hoang et al., 2004; Kuphal et al., 2006; Nozaki et al., 2001; Sakaguchi et al., 2009; Hsieh et al., 2004). However, other data in head and neck cancer and other tumors suggests that DKK3 increases cancer aggressiveness (Hoang et al., 2004; Katase et al., 2012; Nakamura et al., 2007; Wu et al., 2000). Reports on the signaling mechanisms of DKK3 are similarly inconsistent with studies showing either no effect, potentiation or inhibition of Wnt (Hoang et al., 2004; Nakamura et al., 2007; Caricasole et al., 2003).

Recent reports have demonstrated an immuno-modulatory role for DKK3, including induction of CD8+ T-cell tolerance. Exogenous DKK3 inhibited T-cell activity and when DKK3 function was blocked, CD8 T-cell proliferation and IL-2 production was restored (Meister et al., 2015; Lu et al., 2015). However, the precise role of DKK3 in the tumor immune response is far from clear as conflicting reports also describe an immune-stimulatory effect of DKK3 in lung and pancreatic cancer models (Suzawa et al., 2017; Uchida et al., 2015; Uchida et al., 2016).

### II. Aspects of the Present Invention

Herein, DKK3 was identified as a protein expressed in nearly all human PDACs, and in an autochthonous model of PDAC, DKK3 was also present in CP and premalignant PanIN lesions. DKK3 is a secreted factor produced by PSCs in the tumor-associated stroma of PDAC and acts in both an autocrine and paracrine manner, not only to stimulate PSC activity but also to increase PDAC cell proliferation, migration, and invasion. In addition, DKK3 protects cancer cells from undergoing apoptosis induced by chemotherapy. These effects are mediated, at least in part, by NF-κB activation in both PSCs and PDAC cells. Inhibition of DKK3 in xenograft and syngeneic models of PDAC by both genetic ablation and pharmacologic depletion using mAb resulted in inhibition of tumor growth, metastases, improvement in response to chemotherapy and prolongation of survival. These data provide the first evidence that DKK3 acts as a tumor promoter in PDAC and appears to be a promising therapeutic target.

However, ablation of DKK3 did not result in a complete cure since all DKK3-null mice had pancreatic tumors when they died, although the tumors were smaller, less proliferative, and with less active stroma, which likely contributed to their prolonged survival. As a therapeutic approach, the combination of DKK3-targeted therapy with other therapies, including chemotherapy, targeted agents or immunotherapy may result in a more durable response than DKK3 neutralization alone. Another potential application of DKK3 targeting is to intervene at early stages of PDAC development. KPC/DKK3^{-/-} mice had essentially normal pancreata compared with control littermates who had their maximal tumor burden at the same age, suggesting that the absence of DKK3 may have affected tumor initiation or progression. DKK3 was also expressed during the PanIn stage of development in cLGL-Kras^{G12V}/BAC Ela-CreERT mice, which suggests that DKK3 may be involved at an early timepoint. As such, targeting DKK3 at an early stage of PDAC development could be an effective preventive strategy.

Other reports describe DKK3 as a tumor suppressor, and indeed, adenoviral vector delivery of DKK3 has been proposed as a novel treatment approach in xenograft models of prostate, testicular, breast, gastric, and even PDAC (Abarzua et al., 2005; Edamura et al., 2007; Hoang et al., 2004; Kuphal et al., 2006; Nozaki et al., 2001; Sakaguchi et al., 2009; Hsieh et al., 2004; Kawasaki et al., 2009; Tanimoto et al., 2007; Than et al., 2011; Uchida et al., 2014). However, the models used in those studies lacked stromal elements, whereas stromal fibroblasts are the primary source of DKK3 in PDAC. When PSCs were absent in the co-injection orthotopic model of PDAC, DKK3 blocking antibodies had no effect on tumor progression. DKK3 also has a stimulatory effect on prostate stromal cells and retinal ganglion and Muller glia cells (Nakamura et al., 2007; Nakamura & Hackam, 2010; Zenzmaier et al., 2013). In the retina, DKK3 potentiates Wnt signaling, which parallels the observations in PSCs in PDAC (Nakamura et al., 2007). It is conceivable that DKK3 has diverse and even conflicting roles in tumor progression that are cell context-dependent, similar to what is known about TGFβ (Padua & Massague, 2009).

The tumor-associated stroma in various malignancies including PDAC can contribute to an immunosuppressive microenvironment. Kaneda et al. (2016) showed that macrophage lipid kinase PI3Kγ promotes an immunosuppressive tumor microenvironment in PDAC resulting in tumor progression, metastasis, and fibrosis. Inhibition of PI3Kγ restored an antitumor immune response and decreased tumor growth with improved survival. Focal adhesion kinase (FAK) has also been shown to be correlated with low levels of CD8+ T cell infiltration and fibrosis in human PDAC samples (Jiang et al., 2016) and treatment with a FAK inhibitor resulted in decreased tumor growth with improved survival in the KPC model of PDAC. Moreover, FAK inhibition improved responsiveness to T cell immunotherapy and PD-1 inhibitors in the previously unresponsive KPC model. In a similar fashion, DKK3 produced by the PSCs inhibits CD8+ cytotoxic T cells and ablation of DKK3 in the KPC model resulted in a robust infiltration of cytotoxic T cells into the tumors. Tumor inhibition with anti-DKK3 mAb was more effective in the immune-competent syngeneic and GEMMs of PDAC compared to immunodeficient xenograft models, which also suggests that the effects of DKK3 blockade may be amplified in the presence of an intact immune system. The observation that survival was equal in KPC mice with either homozygous or heterozygous depletion of DKK3 was unexpected. Whether partial ablation of DKK3 results in a similar degree of CD8+ T cell recruitment as in DKK3-null mice is unknown and more studies are underway to address this question. What is clear is that checkpoint inhibitor therapy was not effective in the KPC model of PDAC, mirroring the results seen in clinical trials. However, DKK3 neutralization with either genetic ablation or pharmacologic blockade with mAb was able to overcome resistance to immunotherapy and significantly prolong survival. The combination of DKK3 blockade with immunotherapy was superior to DKK3 blockade alone to improve survival.

It remains unclear how DKK3 can have such widely pleiotropic effects in various malignancies, as either a tumor suppressor or a tumor promoter. Although DKK3 activity in pancreatic cancer is at least partly dependent on NF-κB activation, the signaling mechanisms have not been fully elucidated and the receptor for DKK3 has not been firmly established. Additional insight on these questions would be important to not only understand the diverse functions of DKK3 but also to improve the specificity of DKK3-targeted therapies in clinical trials to increase their efficacy and minimize toxicities.

In conclusion, DKK3 is frequently expressed in PDAC and promotes tumor progression, metastasis, and chemoresistance that depends at least in part on NF-κB activation. Inhibition of DKK3 by either genetic ablation or pharmacologic mAb blockade was effective in slowing pancreatic tumor growth with a significant improvement in survival. Furthermore, inhibition of DKK3 was able to overcome resistance to immunotherapy with anti-CTLA-4 inhibitor resulting in long-term durable improvement in survival. As such, DKK3 may be a therapeutic target as either monotherapy or in combination with immunotherapy.

### III. Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv)), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single antibody; (vi) the dAb fragment which consists of a VH domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (VH) connected to a light chain variable domain (VL) in the same polypeptide chain; (xi) “linear antibodies” comprising a pair of tandem Fd segments (VH-CH1-VH-CH1) which, together with complementary light chain polypeptides, form a pair of antigen binding regions.

“Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. Typically, in these chimeric antibodies, the variable region of both light and heavy chains mimics the variable regions of antibodies derived from one species of mammals, while the constant portions are homologous to the sequences in antibodies derived from another. For example, the variable regions can conveniently be derived from presently known sources using readily available hybridomas or B cells from non-human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However, the definition is not limited to this particular example.

A “constant region” of an antibody refers to the constant region of the antibody light chain or the constant region of the antibody heavy chain, either alone or in combination. The constant regions of the light chain (CL) and the heavy chain (CH1, CH2 or CH3, or CH4 in the case of IgM and IgE) confer important biological properties such as secretion, transplacental mobility, Fc receptor binding, complement binding, and the like. By convention the numbering of the constant region domains increases as they become more distal from the antigen binding site or amino-terminus of the antibody.

A “variable region” of an antibody refers to the variable region of the antibody light chain or the variable region of the antibody heavy chain, either alone or in combination. The variable regions of both the light (VL) and heavy (VH) chain portions determine antigen recognition and specificity. VL and VH each consist of four framework regions (FR) connected by three complementarity determining regions (CDRs) also known as hypervariable regions. The CDRs complement an antigen's shape and determine the antibody's affinity and specificity for the antigen. There are six CDRs in both VL and VH. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (the Kabat numbering scheme; see Kabat et al., Sequences of Proteins of Immunological Interest (5th ed., 1991, National Institutes of Health, Bethesda Md.)); and (2) an approach based on crystallographic studies of antigen-antibody complexes (the Chothia numbering scheme which corrects the sites of insertions and deletions (indels) in CDR-L1 and CDR-H1 suggested by Kabat; see Al-lazikani et al. (1997) J. Molec. Biol. 273:927-948)). Other numbering approaches or schemes can also be used. As used herein, a CDR may refer to CDRs defined by either approach or by a combination of both approaches or by other desirable approaches. In addition, a new definition of highly conserved core, boundary and hyper-variable regions can be used.

The term “heavy chain” as used herein refers to the larger immunoglobulin subunit which associates, through its amino terminal region, with the immunoglobulin light chain. The heavy chain comprises a variable region (VH) and a constant region (CH). The constant region further comprises the CH1, hinge, CH2, and CH3 domains. In the case of IgE, IgM, and IgY, the heavy chain comprises a CH4 domain but does not have a hinge domain. Those skilled in the art will appreciate that heavy chains are classified as gamma, mu, alpha, delta, or epsilon (γ, μ, α, δ, ε), with some subclasses among them (e.g., γ1-γ4). It is the nature of this chain that determines the “class” of the antibody as IgG, IgM, IgA IgG, or IgE, respectively. The immunoglobulin subclasses (isotypes), e.g., IgG1, IgG2, IgG3, IgG4, IgA1, etc. are well characterized and are known to confer functional specialization.

The term “light chain” as used herein refers to the smaller immunoglobulin subunit which associates with the amino terminal region of a heavy chain. As with a heavy chain, a light chain comprises a variable region (VL) and a constant region (CL). Light chains are classified as either kappa or lambda (κ, λ). A pair of these can associate with a pair of any of the various heavy chains to form an immunoglobulin molecule. Also encompassed in the meaning of light chain are light chains with a lambda variable region (V-lambda) linked to a kappa constant region (C-kappa) or a kappa variable region (V-kappa) linked to a lambda constant region (C-lambda).

“Nucleic acid,” “nucleic acid sequence,” “oligonucleotide,” “polynucleotide” or other grammatical equivalents as used herein means at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The terms “peptide,” “polypeptide” and “protein” used herein refer to polymers of amino acid residues. These terms also apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. In the present case, the term “polypeptide” encompasses an antibody or a fragment thereof.

Other terms used in the fields of recombinant nucleic acid technology, microbiology, immunology, antibody engineering, and molecular and cell biology as used herein will be generally understood by one of ordinary skill in the applicable arts.

### IV. Antibodies and Modifications of Antibodies

In one embodiment, the antibody is a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557, incorporated herein by reference). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024, each incorporated herein by reference.

In certain embodiments, are antibody conjugates. The conjugate can be, for example, a specific binding agent (such as an antibody) of the invention conjugated to other proteinatious, carbohydrate, lipid, or mixed moiety molecule(s). Such antibody conjugates include, but are not limited to, modifications that include linking it to one or more polymers. In certain embodiments, an antibody is linked to one or more water-soluble polymers. In certain such embodiments, linkage to a water-soluble polymer reduces the likelihood that the antibody will precipitate in an aqueous environment, such as a physiological environment. In certain embodiments, a therapeutic antibody is linked to a water-soluble polymer. In certain embodiments, one skilled in the art can select a suitable water-soluble polymer based on considerations including, but not limited to, whether the polymer/antibody conjugate will be used in the treatment of a patient and, if so, the pharmacological profile of the antibody (e.g., half-life, dosage, activity, antigenicity, and/or other factors).

In further embodiments, the conjugate can be, for example, a cytotoxic agent. Cytotoxic agents of this type may improve antibody-mediated cytotoxicity, and include such moieties as cytokines that directly or indirectly stimulate cell death, radioisotopes, chemotherapeutic drugs (including prodrugs), bacterial toxins (e.g., pseudomonas exotoxin, diphtheria toxin, etc.), plant toxins (e.g., ricin, gelonin, etc.), chemical conjugates (e.g., maytansinoid toxins, calechaemicin, etc.), radioconjugates, enzyme conjugates (e.g., RNase conjugates, granzyme antibody-directed enzyme/prodrug therapy), and the like. Protein cytotoxins can be expressed as fusion proteins with the specific binding agent following ligation of a polynucleotide encoding the toxin to a polynucleotide encoding the binding agent. In still another alternative, the specific binding agent can be covalently modified to include the desired cytotoxin.

In additional embodiments, antibodies, or fragments thereof, can be conjugated to a reporter group, including, but not limited to a radiolabel, a fluorescent label, an enzyme (e.g., that catalyzes a colorimetric or fluorometric reaction), a substrate, a solid matrix, or a carrier (e.g., biotin or avidin). The invention accordingly provides a molecule comprising an antibody molecule, wherein the molecule preferably further comprises a reporter group selected from the group consisting of a radiolabel, a fluorescent label, an enzyme, a substrate, a solid matrix, and a carrier. Such labels are well known to those of skill in the art, e.g., biotin labels are particularly contemplated. The use of such labels is well known to those of skill in the art and is described in, e.g., U.S. Pat. Nos. 3,817,837; 3,850,752; 3,996,345 and 4,277,437, each incorporated herein by reference. Other labels that will be useful include but are not limited to radioactive labels, fluorescent labels and chemiluminescent labels. U.S. Patents concerning use of such labels include for example U.S. Pat. Nos. 3,817,837; 3,850,752; 3,939,350 and 3,996,345. Any of the peptides of the present invention may comprise one, two, or more of any of these labels.

A. Monoclonal Antibodies and Production Thereof

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the VH and the C_{L} is aligned with the first constant domain of the heavy chain (C_{H1}). Particular amino acid residues are believed to form an interface between the light chain and heavy chain variable regions. The pairing of a VH and VL together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (CL). Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in CH sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the VL, and 26-32 (H1), 52-56 (H2) and 95-101 (H3) in the VH when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The monoclonal antibodies may also be isolated from phage antibody libraries using the techniques described in Clackson et al., Nature, 352:624-628 (1991) and Marks et al., J. Mol. Biol., 222:581-597 (1991), for example.

B. Humanized Antibodies and Production Thereof

Where the antibodies or their fragments are intended for therapeutic purposes, it may desirable to “humanize” them in order to attenuate any immune reaction. Such humanized antibodies may be studied in an in vitro or an in vivo context. Humanized antibodies may be produced, for example by replacing an immunogenic portion of an antibody with a corresponding, but non-immunogenic portion (i.e., chimeric antibodies). PCT Application PCT/US86/02269; EP Application 184,187; EP Application 171,496; EP Application 173,494; PCT Application WO 86/01533; EP Application 125,023; Sun et al. (1987); Wood et al. (1985); and Shaw et al. (1988); all of which references are incorporated herein by reference. General reviews of “humanized” chimeric antibodies are provided by Morrison (1985; also incorporated herein by reference. “Humanized” antibodies can alternatively be produced by CDR or CEA substitution. Jones et al. (1986) and Beidler et al. (1988), each of which is incorporated herein by reference. For this, human VH and VL sequences homologous to the VH and VL frameworks of the mouse monoclonal antibody can be identified by searching within the GenBank database. The human sequences with the highest homology are then was chosen as an acceptor for humanization. The CDR sequences of mouse monoclonal are then transferred to the corresponding positions of selected human frameworks.

C. General Methods

It will be understood that monoclonal antibodies of the present invention have several applications. These include the production of diagnostic kits for use in detecting DKK3, as well as for treating diseases associated with increased levels of DKK3. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce antigen-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

Methods for generating hybrids of antibody-producing cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

In another aspect, there are provided monoclonal antibodies having clone-paired CDRs from the heavy and light chains as illustrated in Tables 1 and 2, respectively. Such antibodies may be produced using methods described herein.

In another aspect, the antibodies may be defined by their variable sequence, which include additional “framework” regions. These are provided in Tables 3 and 4 that encode or represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (1) the amino acids may vary from those set out above by permitting conservative substitutions (discussed below). Each of the foregoing applies to the nucleic acid sequences set forth as Table 3 and the amino acid sequences of Table 4.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1 - - - 6) Dextran Increases Its Affinity For Antigen,” J. Exp. Med. 168(3): 1099-1109; Tao, M. H. et al. (1989) “Studies Of Aglycosylated Chimeric Mouse-Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with GO, G1F, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication 20030003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Antibodies can be engineered for enhanced biophysical properties. One can use elevated temperature to unfold antibodies to determine relative stability, using average apparent melting temperatures. Differential Scanning calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Generally, it is thought that autoreactive clones should be eliminated during ontogeny by negative selection; however it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### V. Treatment of Disease

Certain aspects of the present embodiments can be used to prevent or treat a disease or disorder associated with elevated levels of DKK3, such as cancer, such as pancreatic ductal adenocarcinoma or breast cancer. Functioning of DKK3 may be reduced by any suitable drugs. Preferably, such substances would be an anti-DKK3 antibody.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that inhibits the DKK3, either alone or in combination with administration of chemotherapy, immunotherapy, or radiotherapy, performance of surgery, or any combination thereof.

The term “subject” as used herein refers to any individual or patient to which the subject methods are performed. Generally, the subject is human, although as will be appreciated by those in the art, the subject may be an animal. Thus, other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, a neurodegenerative disease, and/or a genetic disorder).

Where clinical application of a therapeutic composition containing an inhibitory antibody is undertaken, it will generally be beneficial to prepare a pharmaceutical or therapeutic composition appropriate for the intended application. In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The active compounds can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain embodiments, the compositions and methods of the present embodiments involve an antibody or an antibody fragment against DKK3, in combination with a second or additional therapy, such as chemotherapy or immunotherapy. Such therapy can be applied in the treatment of any disease that is associated with elevated DKK3. For example, the disease may be a cancer.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an antibody therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PS Kpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; PARP inhibitors, such as olaparib, rucaparib, niraparib, talazoparib, BMN673, iniparib, CEP 9722, or ABT888 (veliparab); CDK4/6 inhibitors, such as ribociclib, palbociclib, ademaciclib, or trilaciclib; androgen inhibitor and anti-androgens, such as cyproterone acetate, megestrol acetate, chlormadinone acetate, spironolactone, oxendolone, osaterone acetate, flutamide, bicalutamide, nilutamide, topilutamide, enzalutamide, apalutamide, dienogest, drospirenone, medrogestone, nomegestrol acetate, promegestone, trimegeston, ketoconazole, abiraterone acetate, seviteronel, aminoglutethimide, finasteride, dutasteride, epristeride, alfatradiol, saw palmetto extract (Serena repens), medrogestone, and bifluranol; carboplatin, procarbazine, plicomycin, gemcitabien, navelbine, farnesyl-protein tansferase inhibitors, transplatinum, and pharmaceutically acceptable salts, acids, or derivatives of any of the above.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Immune checkpoints either turn up a signal (e.g., co-stimulatory molecules) or turn down a signal Immune checkpoint proteins that may be targeted by immune checkpoint blockade include adenosine A2A receptor (A2AR), B7-H3 (also known as CD276), B and T lymphocyte attenuator (BTLA), CCLS, CD27, CD38, CD8A, CMKLR1, cytotoxic T-lymphocyte-associated protein 4 (CTLA-4, also known as CD152), CXCL9, CXCR5, glucocorticoid-induced tumour necrosis factor receptor-related protein (GITR), HLA-DRB1, ICOS (also known as CD278), HLA-DQA1, HLA-E, indoleamine 2,3-dioxygenase 1 (IDO1), killer-cell immunoglobulin (KIR), lymphocyte activation gene-3 (LAG-3, also known as CD223), Mer tyrosine kinase (MerTK), NKG7, OX40 (also known as CD134), programmed death 1 (PD-1), programmed death-ligand 1 (PD-L1, also known as CD274), PDCD1LG2, PSMB10, STAT1, T cell immunoreceptor with Ig and ITIM domains (TIGIT), T-cell immunoglobulin domain and mucin domain 3 (TIM-3), V-domain Ig suppressor of T cell activation (VISTA, also known as C10orf54), and 4-1BB (CD137). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardoll, Nat Rev Cancer, 12(4): 252-264, 2012; both incorporated herein by reference). Known inhibitors of the immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95(17): 10067-10071; Camacho et al. (2004) J Clin Oncology, 22(145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T cell activation (VISTA), also known as C10orf54. The complete protein sequence of human VISTA has the Genbank accession number NP_071436. VISTA is found on white blood cells and inhibits T cell effector function. In some embodiments, the immune checkpoint inhibitor is an anti-VISTAS antibody (e.g., a human antibody, a humanized antibody, or a chimeric antibody), an antigen binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is indoleamine 2,3-dioxygenase (IDO). The complete protein sequence of human IDO has Genbank accession number NP_002155. In some embodiments, the immune checkpoint inhibitor is a small molecule IDO inhibitor. Exemplary small molecules include BMS-986205, epacadostat (INCB24360), and navoximod (GDC-0919).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen binding fragment thereof, an immunoadhesin, a fusion protein, or oligopeptide. Anti-human-ICOS antibodies (or VH and/or VL domains derived therefrom) suitable for use in the present methods can be generated using methods well known in the art. Alternatively, art recognized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Another immune checkpoint protein that can be targeted in the methods provided herein is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art recognized anti-TIGIT antibodies can be used. An exemplary anti-TIGIT antibody is MK-7684 (see, e.g., WO 2017/030823, WO 2016/028656).

Another immune checkpoint protein that can be targeted in the methods provided herein is OX40, also known as CD134. The complete protein sequence of human OX40 has Genbank accession number NP_003318. In some embodiments, the immune checkpoint inhibitor is an anti-OX40 antibody (e.g., a human antibody, a humanized antibody, or a chimeric antibody), an antigen binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another immune checkpoint protein that can be targeted in the methods provided herein is glucocorticoid-induced tumour necrosis factor receptor-related protein (GITR), also known as TNFRSF18 and AITR. The complete protein sequence of human GITR has Genbank accession number NP_004186. In some embodiments, the immune checkpoint inhibitor is an anti-GITR antibody (e.g., a human antibody, a humanized antibody, or a chimeric antibody), an antigen binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art recognized anti-GITR antibodies can be used. An exemplary anti-GITR antibody is TRX518 (see, e.g., WO 2006/105021).

Another immune checkpoint protein that can be targeted in the methods provided herein is T-cell immunoglobulin and mucin-domain containing-3 (TIM3), also known as HAVCR2. The complete protein sequence of human TIM3 has Genbank accession number NP_116171. In some embodiments, the immune checkpoint inhibitor is an anti-TIM3 antibody (e.g., a human antibody, a humanized antibody, or a chimeric antibody), an antigen binding fragment thereof, an immunoadhesin, a fusion protein, or oligopeptide. Anti-human-TIM3 antibodies (or VH and/or VL domains derived therefrom) suitable for use in the present methods can be generated using methods well known in the art. Alternatively, art recognized anti-TIM3 antibodies can be used. Exemplary anti-TIM3 antibodies include LY3321367 (see, e.g., WO 2018/039020), MBG453 (see, e.g., WO 2015/117002) and TSR-022 (see, e.g., WO 2018/085469).

Another immune checkpoint protein that can be targeted in the methods provided herein is 4-1BB, also known as CD137, TNFRSF9, and ILA. The complete protein sequence of human 4-1BB has Genbank accession number NP_001552. In some embodiments, the immune checkpoint inhibitor is an anti-4-1BB antibody (e.g., a human antibody, a humanized antibody, or a chimeric antibody), an antigen binding fragment thereof, an immunoadhesin, a fusion protein, or oligopeptide. Anti-human-4-1BB antibodies (or VH and/or VL domains derived therefrom) suitable for use in the present methods can be generated using methods well known in the art. Alternatively, art recognized anti-4-1BB antibodies can be used. An exemplary anti-4-1BB antibody is PF-05082566 (utomilumab; see, e.g., WO 2012/032433).

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering (Park, Rosenberg et al. 2011). Isolation and transfer of tumor specific T cells has been shown to be successful in treating melanoma. Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors (Jena, Dotti et al. 2010).

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. Kits

In various aspects of the embodiments, a kit is envisioned containing therapeutic agents and/or other therapeutic and delivery agents. In some aspects, the present embodiments contemplate a kit for preparing and/or administering a therapy of the embodiments. The kit may comprise one or more sealed vials containing any of the pharmaceutical compositions of the present embodiments. The kit may include, for example, at least one DKK3 antibody as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

Cell culture. Cells were confirmed Mycoplasma-free before experiments. HPSCs were developed, and have been described previously (Hwang et al., 2008), from residual PDAC surgical tissue in accordance with the policies and practices of the Institutional Review Board and cell purity was determined by immunohistochemistry for α-SMA, vimentin and desmin as well as morphology and positive staining with Oil Red 0. PDAC cell lines were obtained from the American Type Culture Collection (CFPAC, BxPC3, Capan2, MiaPaca2, MPanc96, HS766t, Panc1, SU86.86, ASPC-1, HPAFII, HPAC, CapanI), Dr. I. J. Fidler (L3.6p1, Department of Cancer Biology, The University of Texas MD Anderson Cancer Center), and Dr. C. Logsdon (Panc3, PSN1, Panc48, Panc28) (Li et al., 2003; Frazier et al., 1990; Yamada et al., 1986). MDA1 and MDA2 primary human PDAC cell lines were developed by passage in a murine xenograft model. KPC murine pancreatic cancer cells were isolated from tumors formed in a genetically engineered KPC mouse model of PDAC (Hingorani et al., 2005), kindly provided by Dr. S. Ullrich (Department of Immunology, MD Anderson Cancer Center). MOH cells were obtained from Dr. R. Mohamed (Wayne State University, Detroit, Mich.). All cells were maintained in 10% fetal bovine serum/Dulbecco's modified Eagle's medium at 37° C. in a humidified atmosphere of 5% CO2. HUVEC cells were obtained from American Type Culture Collection and cultured on plates coated with 0.5% Gelatin A in Minimal Essential Media (Thermo Fisher, Waltham, Mass.) containing 15% FBS, 1 mM sodium pyruvate (Sigma, St. Louis, Mo.), 1× vitamin solution (Thermo Fisher, Waltham, Mass.), 1× Non-Essential Amino Acids and 10 ng/mL bFGF (Thermo Fisher, Waltham, Mass.). For co-culture studies, HPSCs and PDAC cells were cultured in 10-cm Transwell coculture system (Corning Incorporated, Lowell, Mass.) and after 96 hours, cells were harvested for RNA isolation.

RT-PCR. Total RNA was isolated from cells with use of the RNeasy mini kit (Qiagen, Valencia, Calif.), and cDNA was synthesized from total RNA with use of the Quantitect reverse transcription kit (Qiagen, Valencia, Calif.). DKK3 transcripts were amplified by using specific primer pairs DKK3: 5′-CGGCTTCTGGACCTCATC-3′/5′-CGGCTTGCACACATACAC-3′. Collagen1 (COL1A1) transcripts were amplified by using specific primer pairs: COL1A1 5′-CATGAGCCGAAGCTAACCCC-3′/5′-GGGACCCTTAGGCCATTGTG-3′. qPCR was performed in an I-cycler IQ multicolor real-time PCR detection system (Bio-Rad, Hercule, Calif.) using 18s (primer pair 5′-GAGCGGTCGGCGTCCCCCAACTTC-3′/5′-GCGCGTGCAGCCCCGGACATCTAA-3′) as the internal control.

Western blotting. To confirm that DKK3 is secreted by HPSCs, CM was collected as described previously (Kalluri & Zeisberg, 2006), protein concentration was measured by Bradford assay (Bio-Rad Laboratories), and 50 μg of protein was loaded onto a sodium dodecyl sulfate-polyacrylamide (SDS-PAGE) gel and blotted against goat anti-human DKK3 antibody (Abcam, Cambridge, Mass.). To evaluate pathways activated by DKK3, HPSCs were serum-starved overnight and treated with 10 μg/ml rhDKK3. Protein lysates were separated by SDS-PAGE and blotted against total and phosphorylated p65 (Cell Signaling), and total and phosphorylated IκBα (Cell Signaling).

Immunohistochemical analysis. Primary rabbit anti-mouse DKK3 antibody was obtained from Proteintech Inc, goat anti-human DKK3 was obtained from Abcam and rabbit anti-goat secondary antibody was obtained from Jackson ImmunoResearch Laboratories. Monoclonal antibodies against mouse α-SMA, Ki67, CD3 and CD8 were purchased from Abcam, ThermoScientific, Santa Cruz and BioLegend respectively. Slides were blindly evaluated and scored by a dedicated GI pathologist (H.W.).

Dkk3 plasma levels by ELISA. Plasma samples from patients with either PDAC or CP (or normal controls) were obtained under an Institutional Review Board-approved protocol. DKK3 levels were detected with the RayBio® Human DKK-3 ELISA Kit (RayBiotech, Inc, Norcross, Ga.) according to manufacturer-provided instructions.

Expression and silencing of DKK3. Human pcDNA3.1/V5-His A-DKK3 construct was kindly provided by Dr. Lin Zhang (Department of Pharmacology & Chemical Biology, University of Pittsburgh Cancer Institute, Pittsburgh Pa.) (Yue et al., 2008).

DKK3 was silenced by lentiviral transfection with shDKK3 (Open Biosystems, Huntsville, Ala.) and by transfection with siDKK3 (Qiagen, Valencia, Calif.). Stable silencing of DKK3 in HPSCs was achieved by co-transfection of lentiviral plasmid control vector or shDKK3 (HPSC-shControl or HPSC-shDKK3) with packaging vectors into 293T cells with lipofectamine 2000 (Invitrogen, Carlsbad, Calif.). Viral supernatant (200 μl) was added to HPSCs with 8 μg/mL of Polybrene (hexadimethrine bromide) in a 6-well plate for 2 days, and stably transduced cells were selected in 1 μg/mL puromycin. Transient silencing of DKK3 in HPSCs and PDAC cells was achieved by transfection with a mixture of 5 nM siDKK3 and 3 μL of Hiperfect agent according to the manufacturer's recommendations (Qiagen, Valencia, Calif.).

Cell-based assays. Cell proliferation was measured by the MTS assay (Promega, Madison, Wis.). Cell migration and invasion studies with stimulation by recombinant human DKK3 (R&D, Minneapolis, Minn.) were performed using 6.5-mm Transwell® with 8.0-μm pore membrane Insert (Corning Incorporated) and BioCoat Matrigel-coated invasion chambers (BD Biosciences, Bedford, Mass.).

To assess growth in soft agar, cells were seeded into low melting point agarose culture dishes and allowed to grow for 14 days. After staining with p-iodonitrotetrazolium violet (Sigma, St. Louis, Mo.), the total number of colonies were counted in 10 high power fields.

DKK3 was silenced in relatively chemoresistant HS766T using siRNA (Qiagen) and overexpressed in chemisensitive L3.6p1 as described. In the presence of gemcitabine, cell viability was determined by soft agar colony formation. Apoptosis was determined by flow cytometry. In brief, cells were fixed in 70% ethanol, washed, and resuspended in 200 μL of staining buffer (50 μg/mL propidium iodide and 50 units/mL RNAse in PBS). Propidium iodide-stained cells were detected with FACS, and sub-G1% was calculated as percentage of apoptotic cells.

Reverse-phase protein assay (RPPA). Primary HPSC cell lines from 2 different patients (HPSC, HPSC-20Aim) were plated in triplicate in 6-well dishes at 5×10⁵ cells per well and treated with either recombinant human DKK3 (10 μg/mL) or PBS for 20 minutes. Cells were lysed, prepared and analyzed at the University of Texas MD Anderson Cancer Center Functional Proteomic core facility (Houston, Tex.) as described on the world wide web at mdanderson.org/education-and-research/resources-for-professionals/scientific-resources/core-facilities-and-services/functional-proteomics-rppa-core/index.html. Serial dilutions of samples were arrayed on nitrocellulose-coated slides and run against 220 validated antibodies.

NF-κB activity. NF-κB activity was determined by using a luciferase reporter assay. Briefly, cells stably expressing a lenti-NF-κB luciferase reporter construct (Arumugam et al., 2006) in a 96-well plate were treated with rhDKK3 (10 μg/mL) in serum-free conditions for 5 hours. Luciferase signal was detected by using D-luciferin firefly potassium salt (Caliper Life Sciences, Hopkinton, Mass.) and the IVIS® imaging system (Xenogen Corp., Alameda, Calif.).

Binding assay. Cell surface binding of DKK3 was performed using flow cytometry. In brief, BxPC3 or L3.6p1 cells (0.5×10⁶) were incubated for 30 min with His-labeled rhDKK3 (10 μg/mL) with or without JM6-6-1 (70 μg/mL). After washing with binding buffer (3% BSA in PBS), cells were stained with 1:100 anti-His antibody (abCam) for 30 min and followed by staining with secondary antibody conjugated with Dylight488 for 20 min. The cells were fixed with 2% paraformaldehyde and were subjected to fluorescent signal detection by BD FACSCalibur.

PDAC mouse models. Nude mice and C57BL/6 mice were obtained from The Jackson Laboratory and maintained in facilities approved by the Association for Assessment and Accreditation of Laboratory Animal Care International in accordance with current regulations and standards of the U.S. Department of Agriculture, Department of Health and Human Services, and NIH. All animal procedures were reviewed and approved by The University of Texas MD Anderson Cancer Center Institutional Animal Care and Use Committee.

An orthotopic nude mouse model of PDAC using BxPC3 cancer cells labeled with firefly luciferase (BxPC3-FL) co-injected with HPSCs has been previously described (Hwang et al., 2008). Mice received intrapancreatic injections of BxPC3-FL (1×10⁶ per mouse) with HPSC-shControl or HPSC-shDKK3 in a tumor-to-stroma ratio of 1:0 or 1:3 in 50-μL of HBSS and tumor growth was monitored using IVIS imaging (Hwang et al., 2008). In addition, a syngeneic immunocompetent DKK3 null model was used, provided by Dr. C. Niehrs (Barrantes Idel et al., 2006), that was injected with murine PDAC cells (termed “KPC cells”) isolated from tumors arising in a GEMM of PDAC (Pdx1-Cre; Kras^{LSL-G12D};Trp53^{R172H/+}) (Hingorani et al., 2005; Hingorani et al., 2003) and labeled with luciferase.

Two GEMMs of PDAC that differ in the pancreas-specific promoter to target oncogenic Kras expression (Hingorani et al., 2005; Hingorani et al., 2003) were used in the experiments to evaluate the effect of DKK3 neutralizing mAb (Pdx1-Cre; Kras^{LSL-G12D}; Trp53^{R172H/+} and P48-Cre; Kras^{LSL-G12D}; Trp53^{fl/fl}). To evaluate the effects of genetic ablation of DKK3 on pancreatic cancer development, DKK3-null mice were crossed to P48-Cre; Kras^{LSL-G12D};Trp53^{R172H} or P48-Cre; Kras^{LSL-G12D}; Trp53^{fl/fl} mice (Hingorani et al., 2005; Hingorani et al., 2003), and the progeny were monitored until they were moribund. Tissue and blood samples were collected after the mice were euthanized. A genetically engineered mouse model of PDAC that expresses high levels of mutant Kras (cLGL-Kras^{G12V}/BAC Ela-CreERT) (Ji et al., 2009) was used to evaluate the expression of DKK3 at various stages of PDAC development.

DKK3-mAb. Neutralizing mAbs to DKK3 were generated by immunizing A/J mice with purified recombinant human DKK-3/His (R&D). Initial screening was performed by using ELISA high-throughput screening, from which more than 30 hybridoma clones showed strong and specific DKK3 binding. After subcloning of anti-DKK3 mAb hybridomas, 12 purified mAbs were further tested with functional assays. Data are shown using two of the most effective clones (JM6-6-1 and JM8-12-1), and a nonspecific IgG1 isotype control clone was used as a negative control.

T cell division assay. Peripheral blood mononuclear cells (PBMC) were obtained from buffy coat by density centrifugation using Histopaque 1077 (Sigma) and CD3⁺ T-cells were isolated using a pan T-cell isolation kit (Miltenyi Biotec). Cells were labeled with CFSE (Invitrogen) and combined 1:1 with CD3/CD28 human T-cell activator beads (Life Technologies) in a 96-well plate. DKK3 or HPSC-CM was added and after 96 hours, cells were stained for CD3, CD4, and CD8 to define lineage and analyzed by flow cytometry. Data is reported as percentage of proliferating CD3⁺ T-cells.

Statistical analysis. Results are shown from at least three independent experiments and presented as the mean±SEM. Data were analyzed with use of the two-tailed Student's t-test, and a significant difference was defined as p<0.05. Survival analysis was performed by using the Kaplan-Meier method (log rank). All statistical analysis was performed by using GraphPad Prism 6.

### Example 1—DKK3 Overexpression in PDAC and TNBC

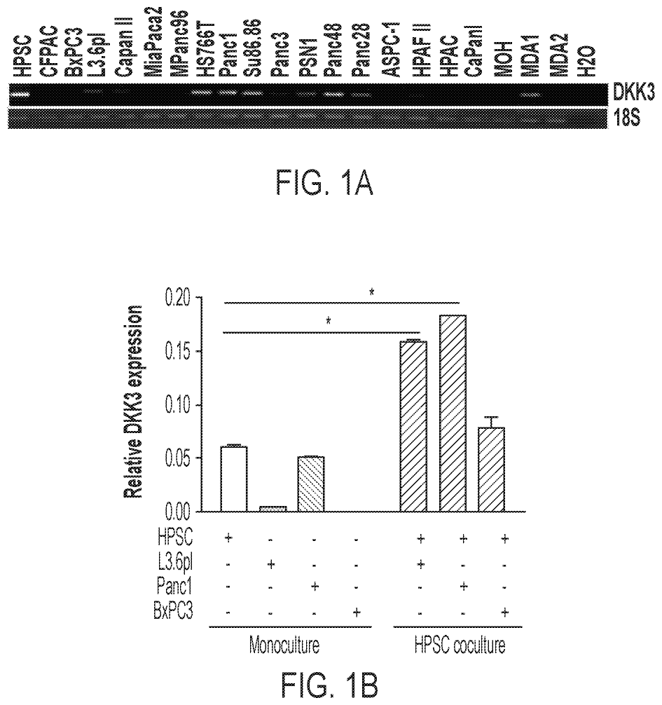

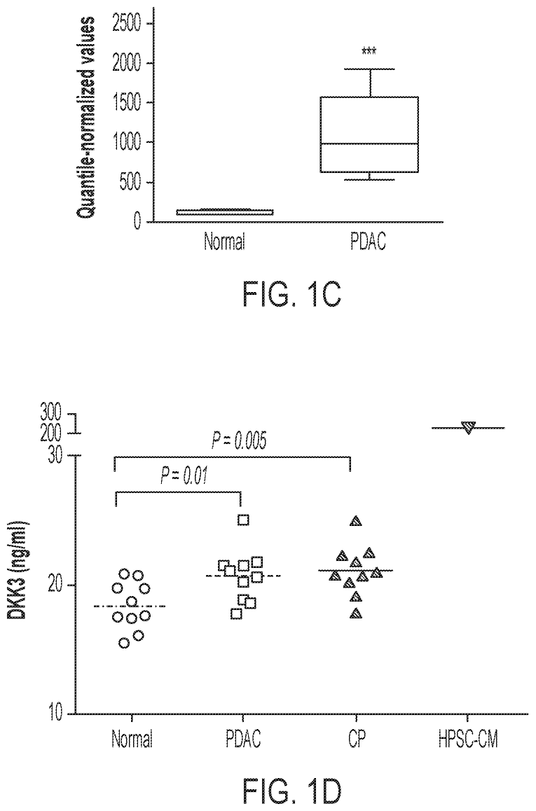

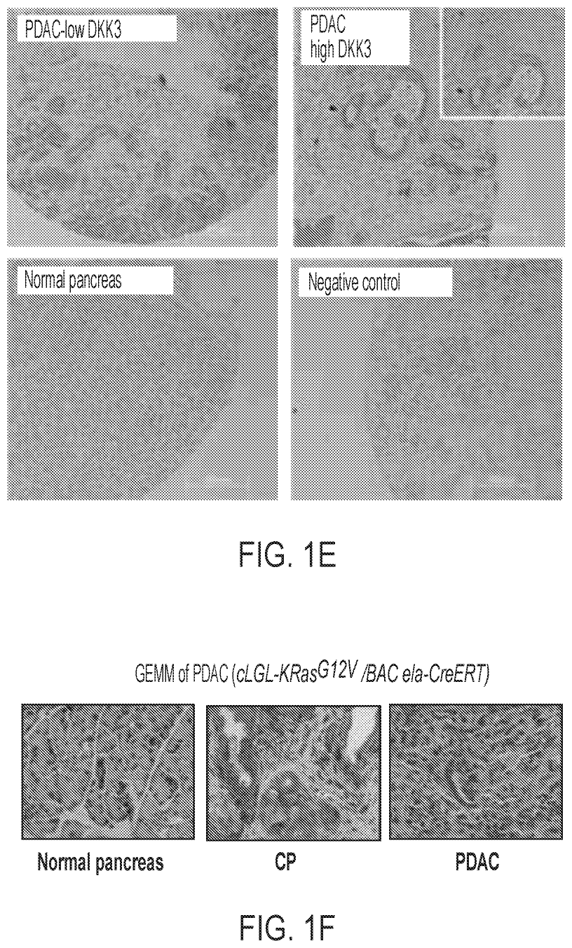

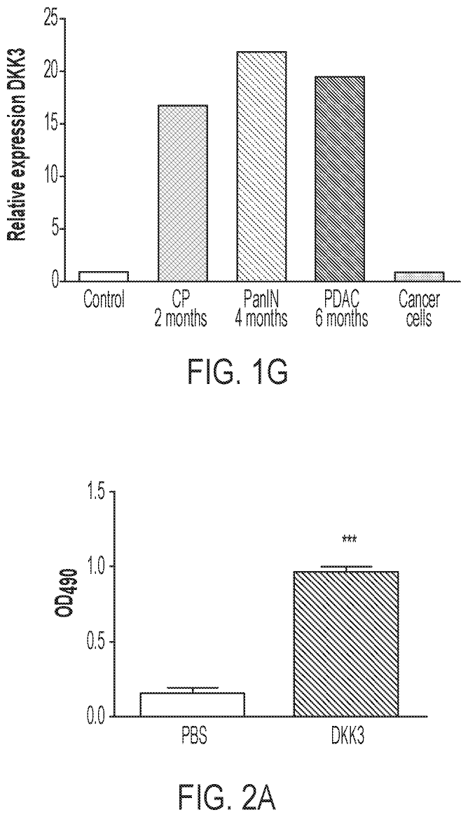

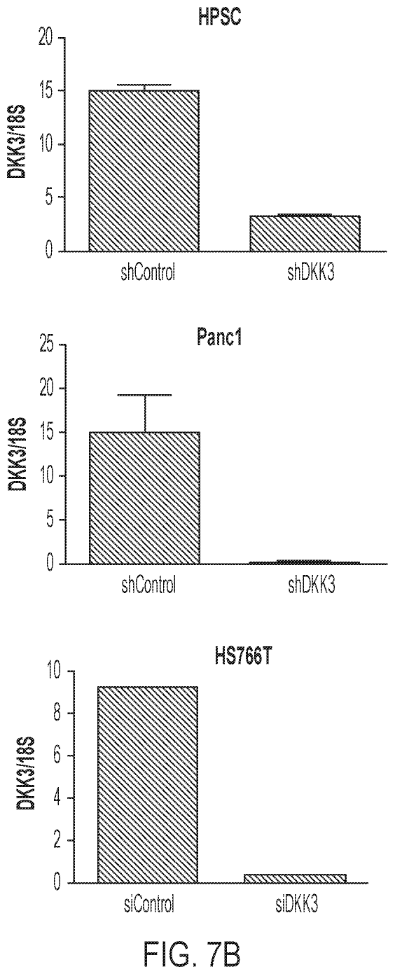

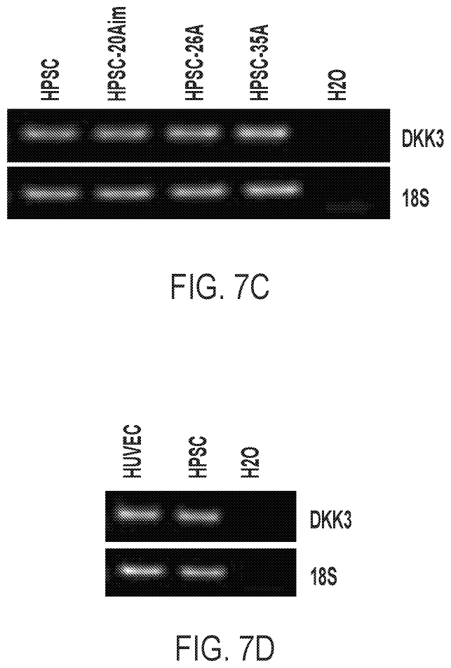

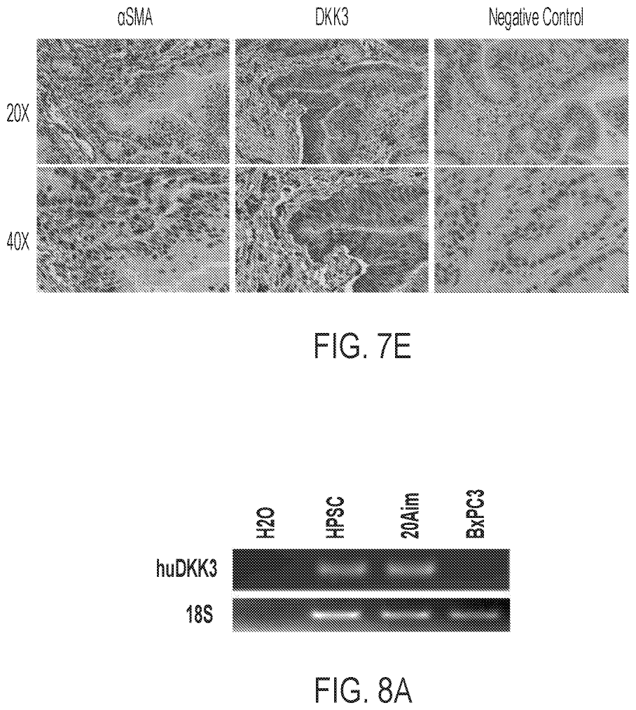

The expression of DKK3 in human PSCs (HPSCs) and 20 PDAC cell lines was examined by reverse transcriptase-polymerase chain reaction (RT-PCR) (FIG. 1A). Expression was strongest in HPSCs with minimal expression in seven cells (HS766T, Panc1, SU86.86, Psn1, Panc48, Panc28, and MDA1) and no expression in the majority (14 of 21) of the cancer cell lines tested. DKK3 is secreted by HPSCs as confirmed by Western blotting of HPSC-conditioned media (HPSC-CM) (FIG. 7A). DKK3 expression was similar in four HPSC preparations from different patients (FIG. 7C).

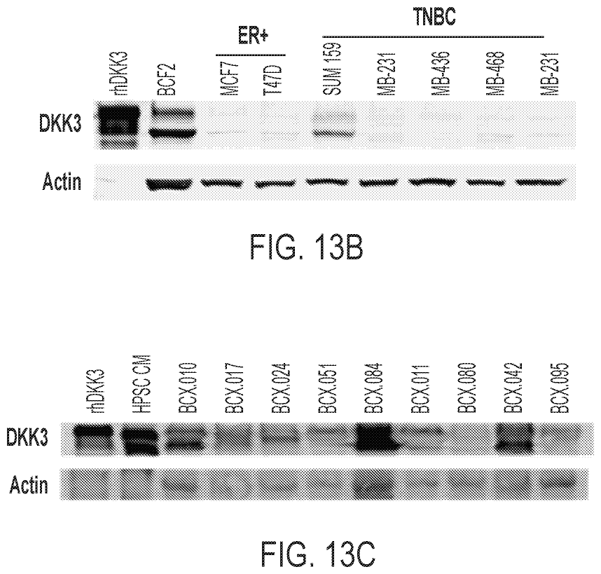

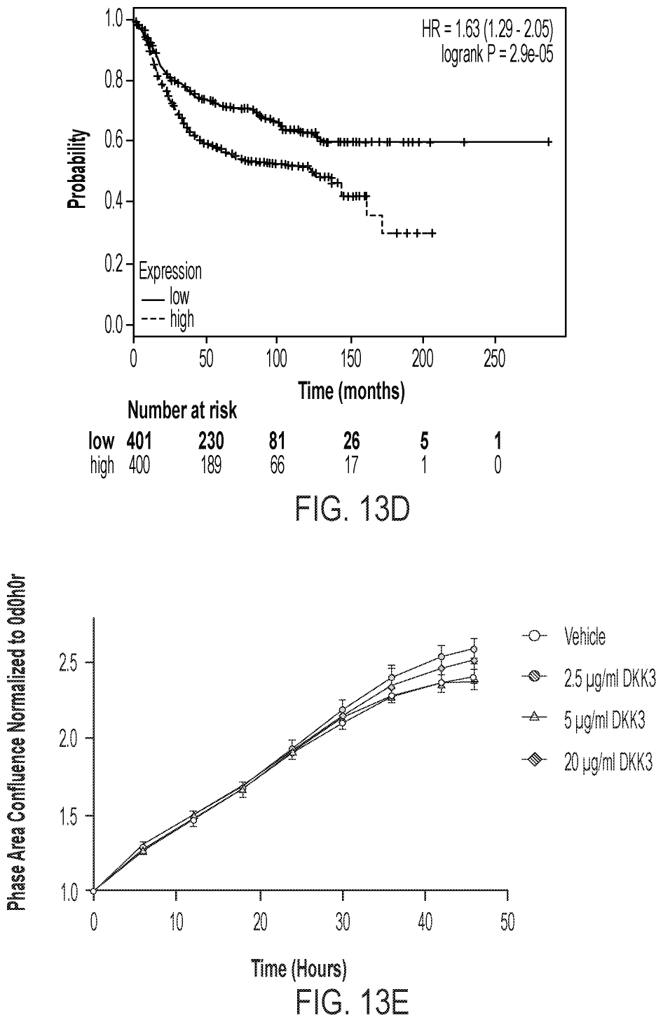

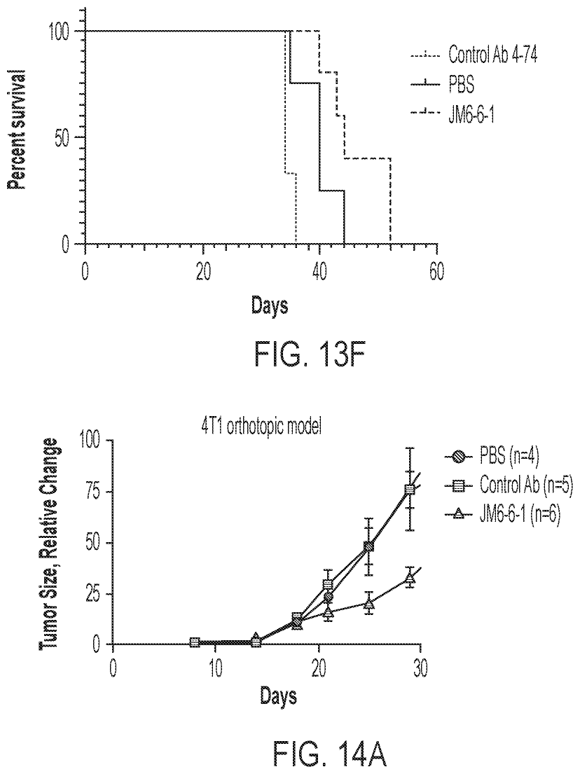

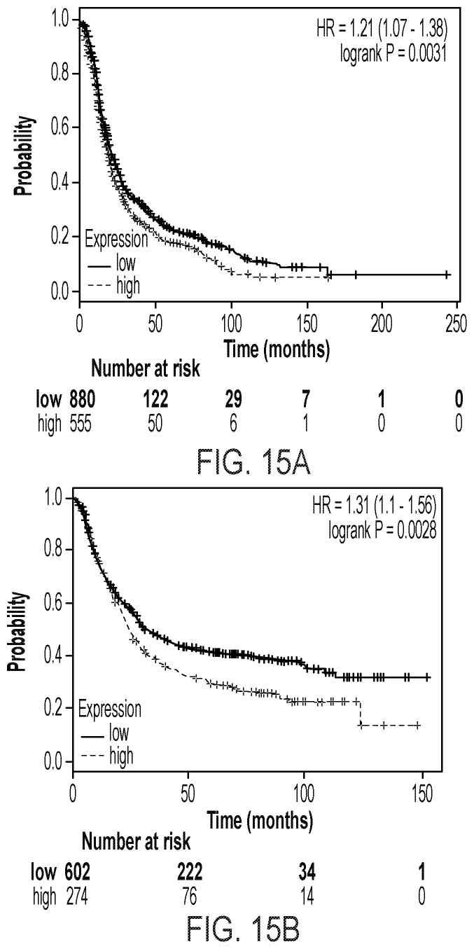

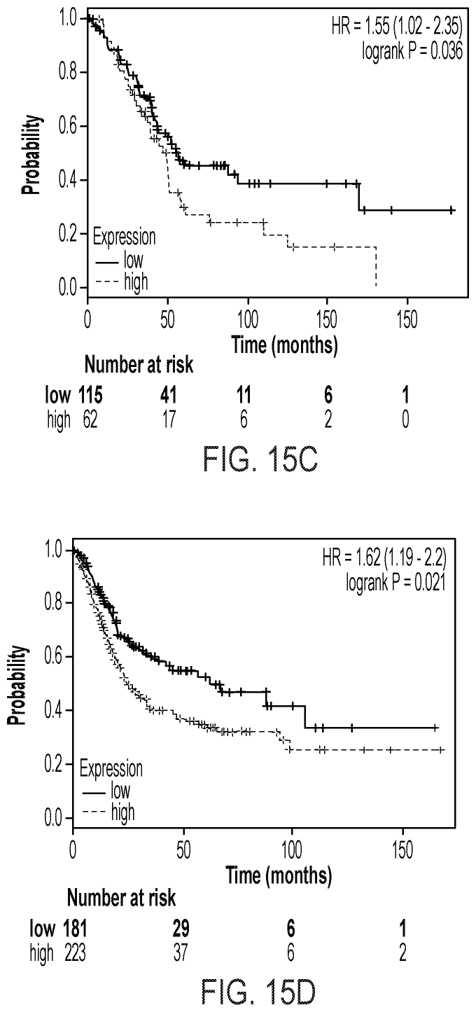

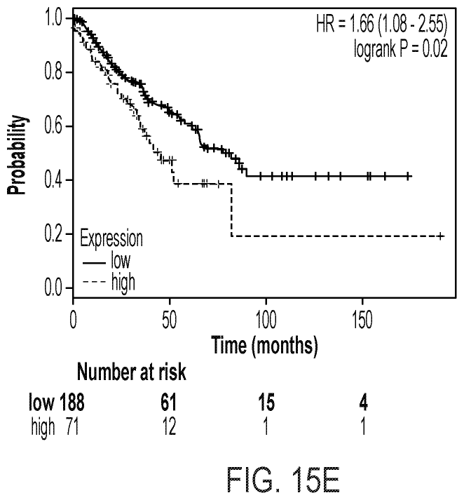

DKK3 expression was also seen at the RNA (FIG. 13A) and protein (FIG. 13B) levels in triple negative breast cancer (TNBC) fibroblasts. DKK3 expression was not seen at the protein level in ER-positive breast cancer cells and in TNBC cancer cell lines (FIG. 13B). It was present in some patient-derived xenograft tumors from TNBC (FIG. 13C). Higher DKK3 expression in various human tumors, including TNBC (FIG. 13D), ovarian cancer (FIG. 15A), gastric cancer (FIG. 15B), PDAC (FIG. 15C), bladder cancer (FIG. 15D), and sarcoma (FIG. 15E), was found to correlate with decreased patient survival. Finally, treatment of SUM159 TNBC cells increased cell proliferation in a dose-dependent manner (FIG. 13E).

Cross-talk between stromal fibroblasts and cancer cells has been described previously (Kalluri & Zeisberg, 2006; Mueller & Fusenig, 2004). Whether coculture of HPSCs and PDAC cells would affect DKK3 expression in either cell population was investigated. Quantitative RT-PCR (qPCR) confirmed minimal to no expression of DKK3 in L3.6p1 and BxPC3 cells in monoculture, whereas expression in Panc1 cells was nearly equivalent to that of HPSCs (FIG. 1B). Coculture of HPSCs with either Panc1 or L3.6p1 cells increased DKK3 expression in HPSCs by 3-fold compared with culturing HPSCs alone (FIG. 1B, white striped bars). There was no increase in DKK3 expression by HPSC after coculture with BxPC3. Conversely, DKK3 expression in the cancer cells was not altered after coculture with HPSCs. Thus, HPSCs express DKK3 at high basal levels, which is further augmented when cocultured with cancer cells, but PDAC cells express minimal levels of DKK3, which does not change with exposure to HPSCs.

DKK3 expression was assessed in human PDAC, and normal pancreas (n=10 per group) using Affymetrix gene expression profiling (Logsdon et al., 2003), which showed 4.5 times higher levels in PDAC than in normal pancreas (FIG. 1C). In a tissue microarray of human PDAC, the expression of DKK3 was confirmed in 118 of 119 samples (99%), with moderate-high expression in 69 samples (58%) (FIG. 1E). Most samples showed DKK3 expressed predominantly in the stroma although several samples with more intense staining demonstrated staining in areas of carcinoma as well. Its expression was not restricted to αSMA-positive cells which is one marker, although not a unique marker, of PSCs (FIG. 7E). DKK3 is expressed in HUVEC cells as well (FIG. 7D). When DKK3 was examined in plasma, PDAC patients had significantly higher mean levels than did healthy volunteers (20.64 ng/mL vs. 18.36 ng/mL, p<0.01; FIG. 1D). DKK3 serum levels were similar in chronic pancreatitis (CP) and PDAC patients. The levels of DKK3 in HPSC-CM were 10 times higher than were levels in the plasma, suggesting that DKK3 may be highly concentrated in the local tumor microenvironment relative to the peripheral circulation.

To evaluate the expression of DKK3 at early stages of PDAC development, a genetically engineered mouse model (GEMM) of PDAC, which expresses high levels of mutant Kras (cLGL-Kras^{G12V}/BAC Ela-CreERT), was examined (Ji et al., 2009). In this model, mice develop CP and early pancreatic intraepithelial neoplasia (PanIN) lesions within 2 months, CP and late PanIN lesions within 4 months, and invasive PDAC with metastases within 6 months. Compared with control mice without pancreatic disease, DKK3 expression was increased 18-fold in CP and early PanINs at 2 months and 21-fold in CP and late PanINs at 4 months (FIGS. 1F & 1G). When mice developed invasive PDAC at 6 months, DKK3 expression was nearly 20 times higher than in controls. In contrast, DKK3 expression was minimal in cancer cells that were isolated from invasive pancreatic tumors formed in this model (FIG. 1G), indicating that DKK3 is primarily derived from stromal cells.

### Example 2—DKK3 Inhibits PDAC and Stellate Cell Activity

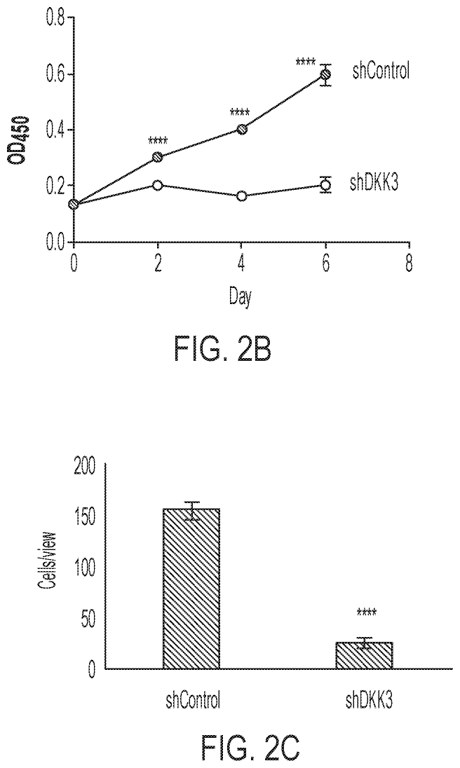

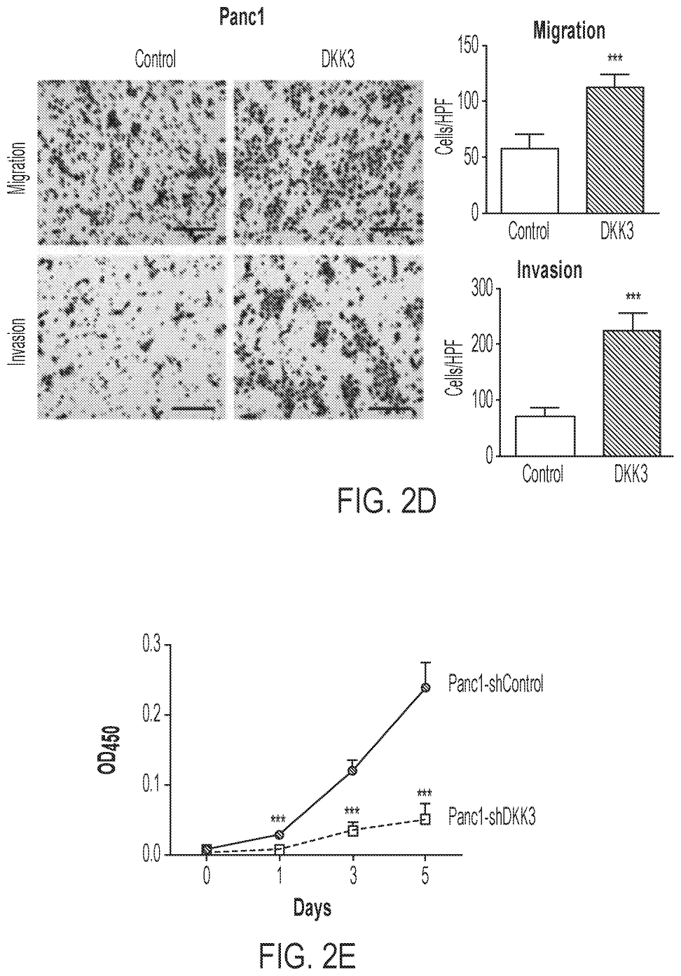

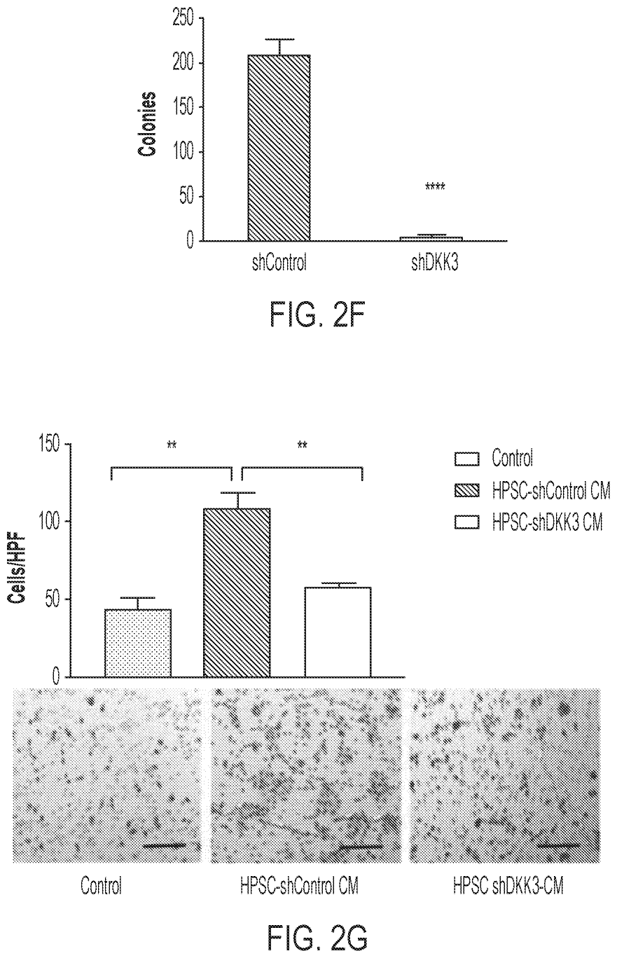

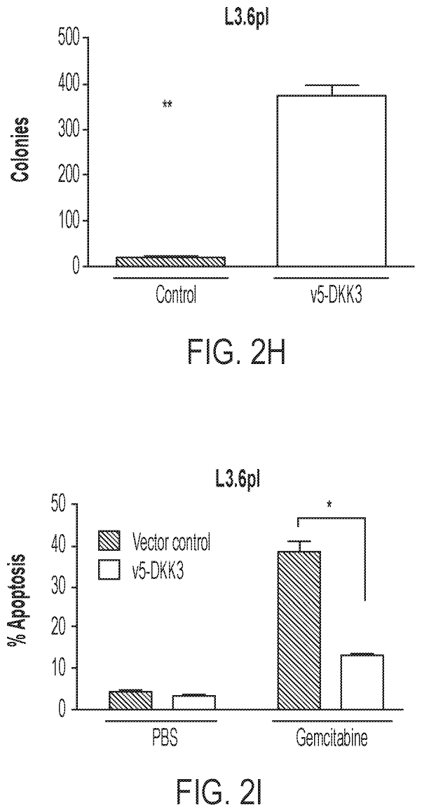

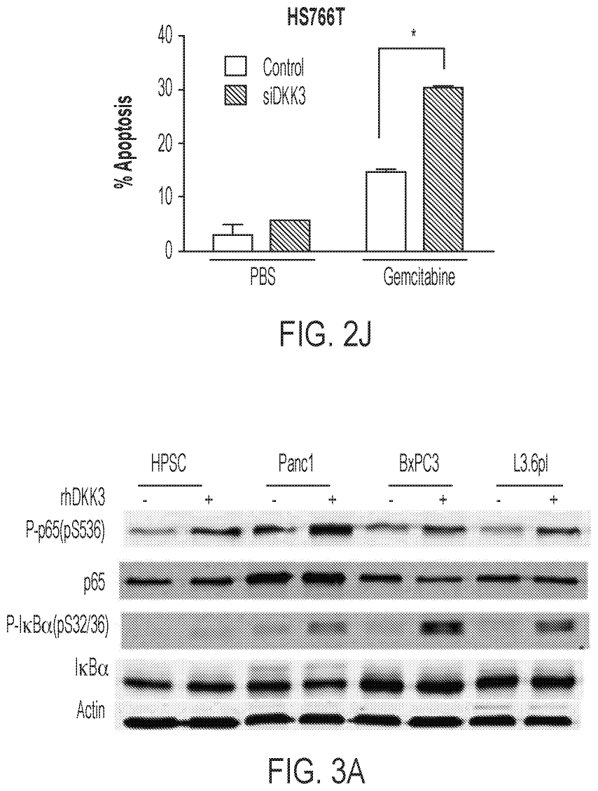

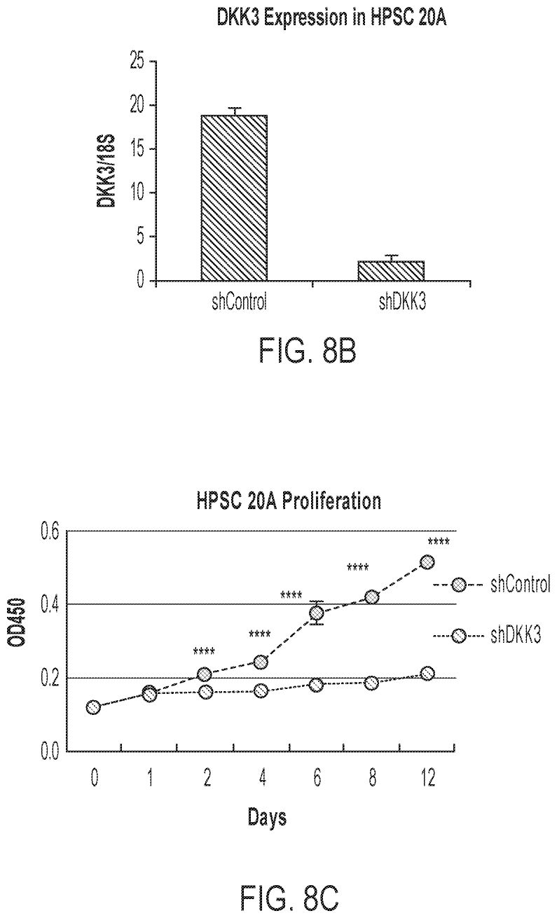

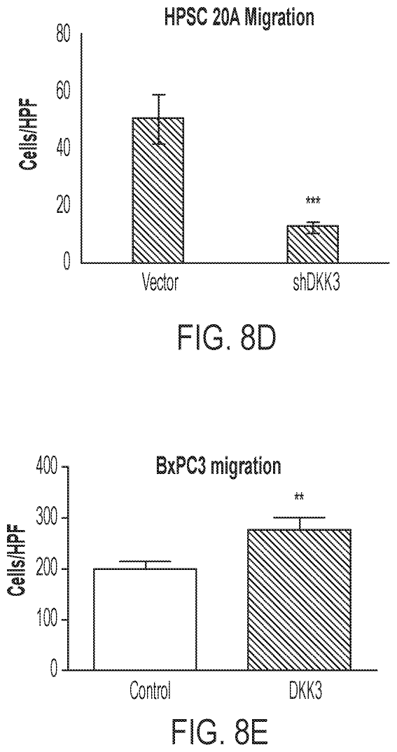

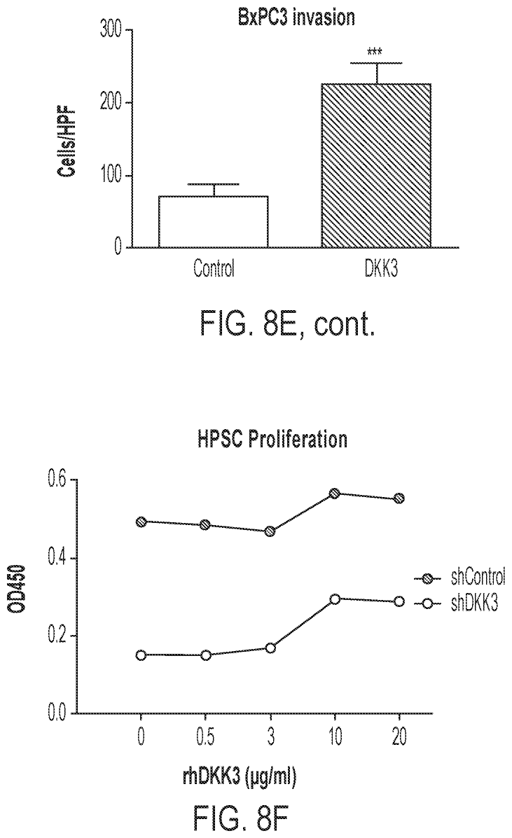

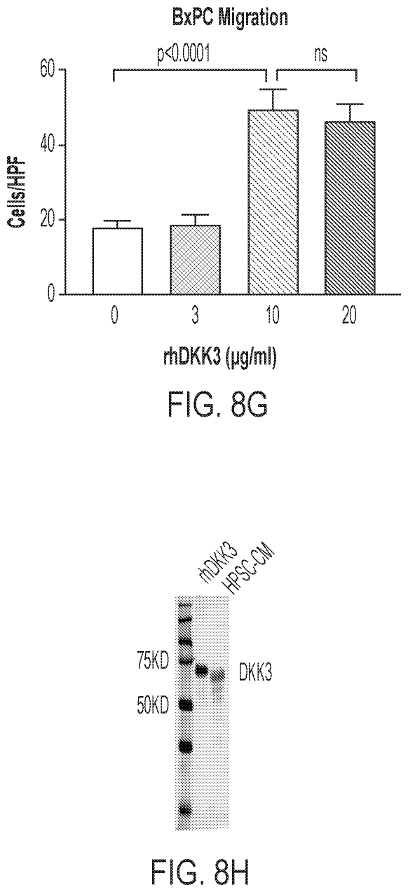

Based on the finding that DKK3 was produced primarily by HPSCs, whether DKK3 had a functional role in HPSC activity was investigated. Treatment with 10 μg/mL of recombinant human DKK3 (rhDKK3) for 48 or 72 hours resulted in significantly increased HPSC proliferation compared with PBS controls (FIG. 2A). Stable silencing of DKK3 in HPSCs (HPSC-shDKK3; FIG. 7B) resulted in a 67% reduction in cell proliferation compared to control cells by day 6 (p<0.00001; FIG. 2B) and a reduction in cell migration to 16.3% of controls (p<0.00001; FIG. 2C). DKK3 expression was confirmed in primary HPSCs from other patients and similar inhibitory effects on proliferation and migration were observed when DKK3 was silenced (FIGS. 8A-D).

Next, whether DKK3 had paracrine effects on PDAC cells was investigated. Treatment of Panc1 cells with rhDKK3 (10 μg/mL) resulted in a nearly 100% increase in migration and more than a 3-fold increase in invasion (p<0.0001 vs. PBS controls; FIG. 2D). Similar results were observed with BxPC3 cells with induction of both migration (p<0.001) and invasion (p<0.0001) compared with controls (FIG. 8E). Results of an initial dose response experiment to test rhDKK3 on HPSC and BxPC3 functional assays are shown in FIGS. 8F-G.

Unlike most other PDAC cell lines, Panc1 expresses a moderate level of DKK3, and stable silencing of DKK3 resulted in inhibition of cell proliferation compared with cells transfected with control shRNA (FIG. 2E) and nearly completely eliminated their ability to grow in soft agar (FIG. 2F), suggesting that DKK3 is critical for anchorage-independent growth.

To confirm whether the effects of DKK3 secreted by HPSCs are similar to those of recombinant DKK3, cells were treated with conditioned medium (CM) from HPSCs transfected with shControl or shDKK3. BxPC3 cells treated with CM from HPSC-shControl showed an 87% increase in migration compared with serum-free media controls whereas migration with CM from HPSC-shDKK3 was similar to media controls (FIG. 2G). A comparison of DKK3 in rhDKK3 and HPSC-CM by Western blotting is shown in FIG. 8H. In summary, DKK3 acts in a paracrine fashion to promote PDAC cell migration, invasion, anchorage-independent growth and, to a lesser degree, proliferation.

### Example 3—DKK3 Induces Resistance to Chemotherapy with Gemcitabine

HPSCs produce secreted factors that enhance chemoresistance in PDAC (Hwang et al., 2008) and therefore, whether DKK3 might contribute to this phenomenon was investigated. L3.6p1 cells are relatively sensitive to gemcitabine and express minimal DKK3, whereas Panc1 and HS766T are relatively resistant to gemcitabine and express a moderate level of DKK3 (Arumugam et al., 2009). When DKK3 was expressed in chemosensitive L3.6p1 cells, colony formation in soft agar in the presence of gemcitabine increased by >90% compared to controls (p<0.001; FIG. 2H), with concomitant reduction in apoptosis (p<0.01; FIG. 2I). DKK3 was transiently silenced in relatively chemoresistant HS766T cells (HS766T-siDKK3) (FIG. 7), and in the presence of gemcitabine, the rate of apoptosis in these cells doubled compared to control cells (p<0.01; FIG. 2J). Taken together, DKK3 contributes to resistance to chemotherapy with gemcitabine.

### Example 4—Autocrine and Paracrine Effects of DKK3 are Mediated by NF-κB Activation

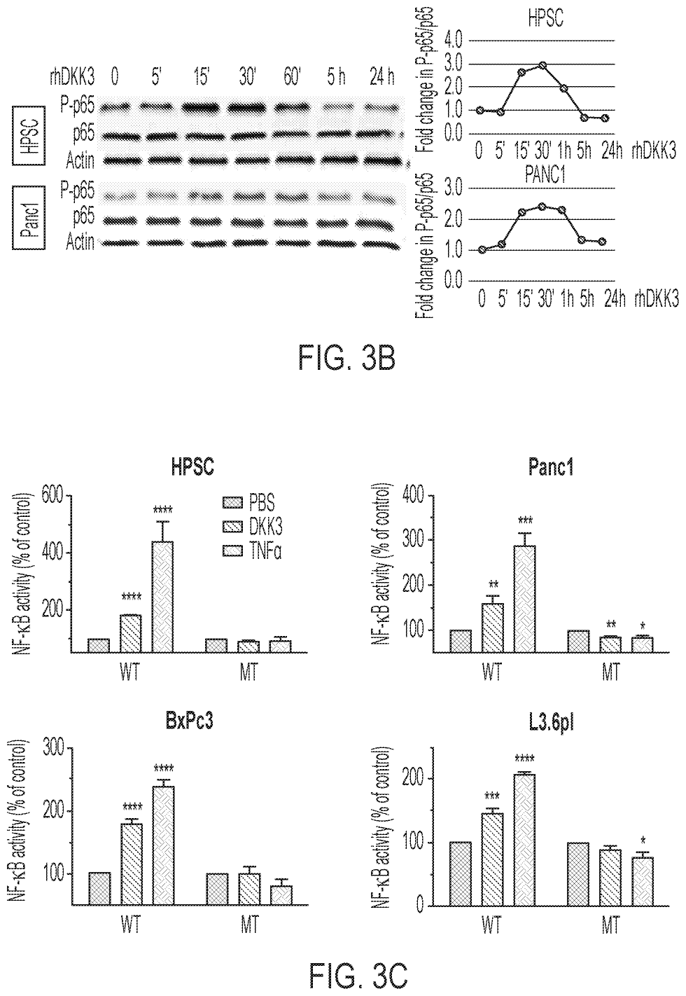

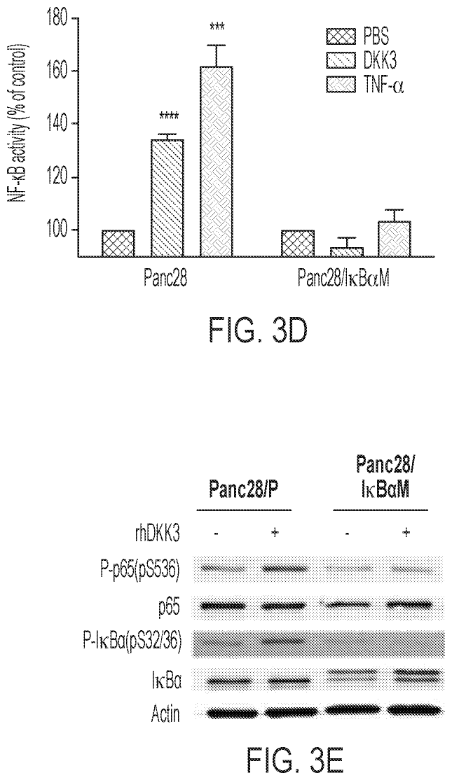

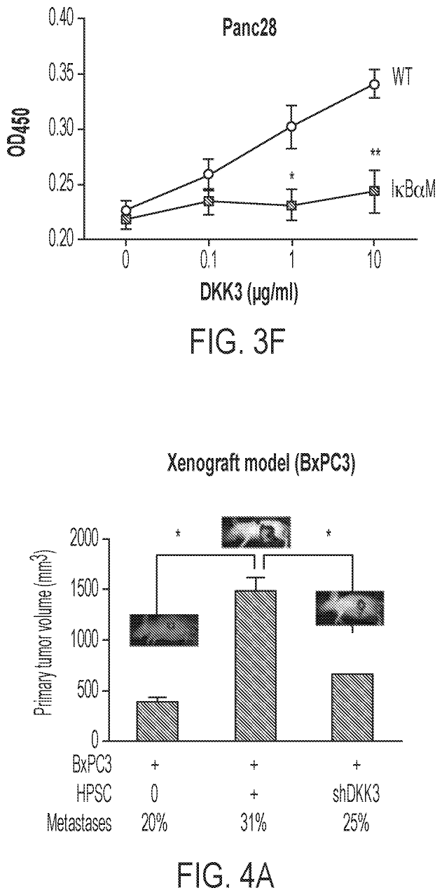

To investigate the mechanisms of DKK3 effects on HPSC and PDAC, high-throughput antibody-based RPPA analysis was performed on primary HPSC cells (HPSC and HPSC20Aim) and PDAC cells (Panc1, BxPC3 and L3.6p1) that were treated with either PBS or rhDKK3 for 20 minutes. Unsupervised clustering revealed that one of the most activated pathways with DKK3 treatment was NF-κB-p65 which was validated with Western blot analysis (FIG. 3A). Stimulation with DKK3 in HPSC and PDAC cells also induced phosphorylation of IκBα, which is regulated by NF-κB, providing additional evidence that DKK3 induced NF-κB activation. Peak phosphorylation of p65 in HPSC and Panc1 occurred at 15-30 minutes after stimulation with rhDKK3 (FIG. 3B). To confirm induction of NF-κB-dependent promoter activity, HPSC, Panc1, BxPC3 and L3.6p1 cells were transfected with either wild-type (WT) or mutant (MT) KB-luciferase reporter gene constructs and stimulated with DKK3. Results of the dual luciferase assay indicated that DKK3 induced NF-κB promoter activity in cells with the wild-type reporter but not in cells with the mutant reporter (FIG. 3C). TNFα was used as a positive control. To further demonstrate the effect of DKK3 on NF-κB activation in PDAC, phosphorylation-defective Panc28 pancreatic cancer cells, which stably express mutated IκBα and are incapable of NF-κB activation (Panc28/IκBαM; gift from Dr. P. Chiao), were used (Niu et al., 2007). Stimulation of parental Panc28 cells with DKK3 induced NF-κB promoter activity by 33% of PBS control whereas no induction was seen in Panc28/IκBαM cells. These results were confirmed by Western blot analysis (FIG. 3E). Finally, whether NF-κB-dependent effects of DKK3 affect PDAC cell function was investigated. Treatment with DKK3 stimulated Panc28 proliferation in a dose-dependent manner. However, proliferation of Panc28/IκBαM was not induced by DKK3, suggesting that NF-κB is required for DKK3-mediated effects on cell proliferation (FIG. 3F). Taken together, these results demonstrate that DKK3 activates NF-κB in both HPSC and PDAC cells and inhibition of NF-κB blocks DKK3-mediated induction of PDAC cell activity.

### Example 5—Silencing of DKK3 in HPSCs Inhibits Tumor Growth In Vivo

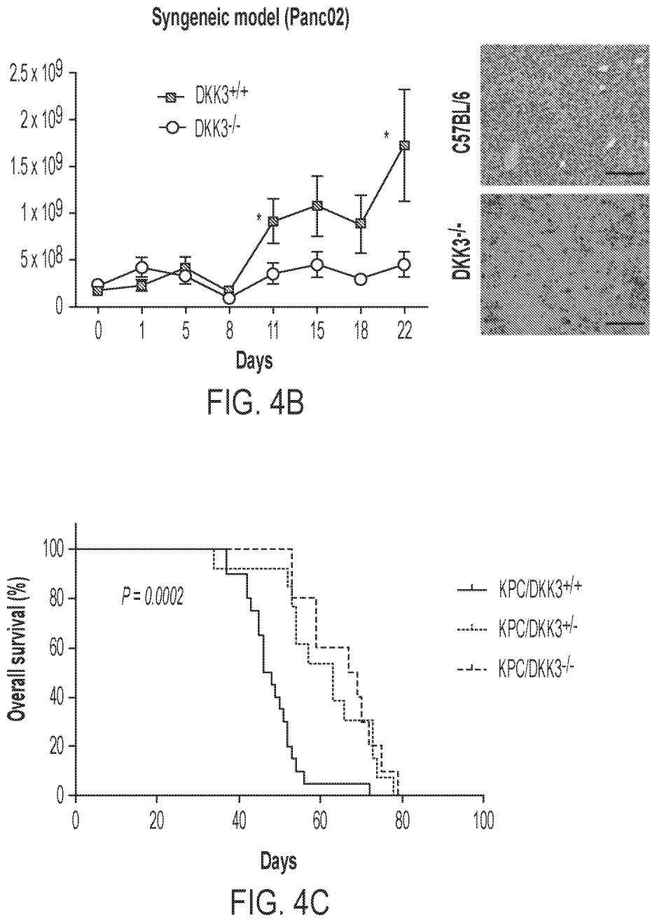

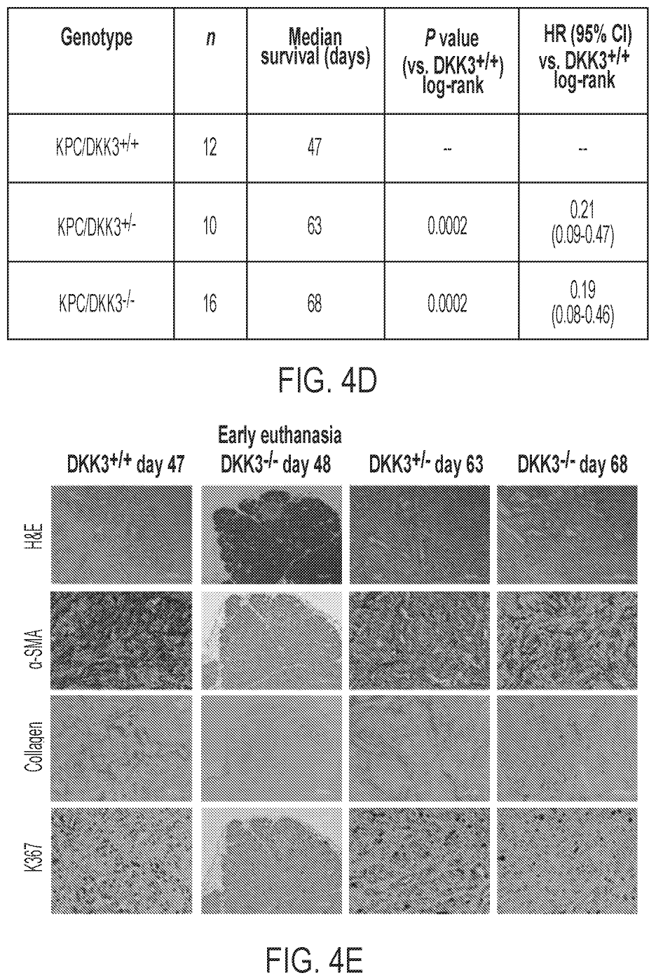

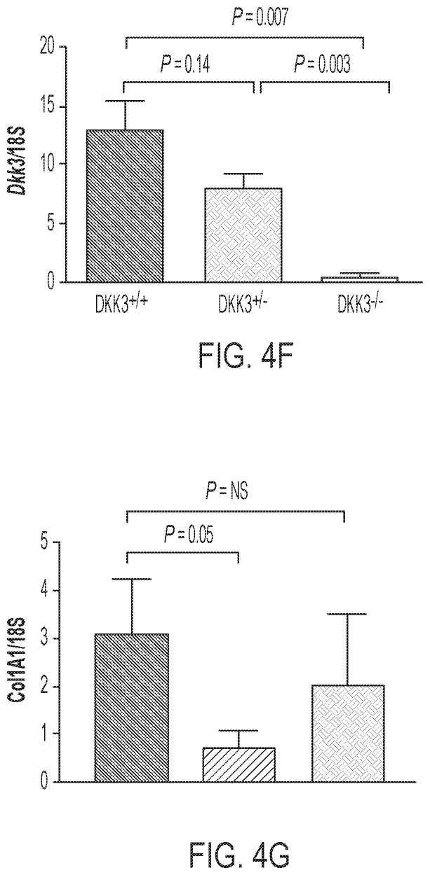

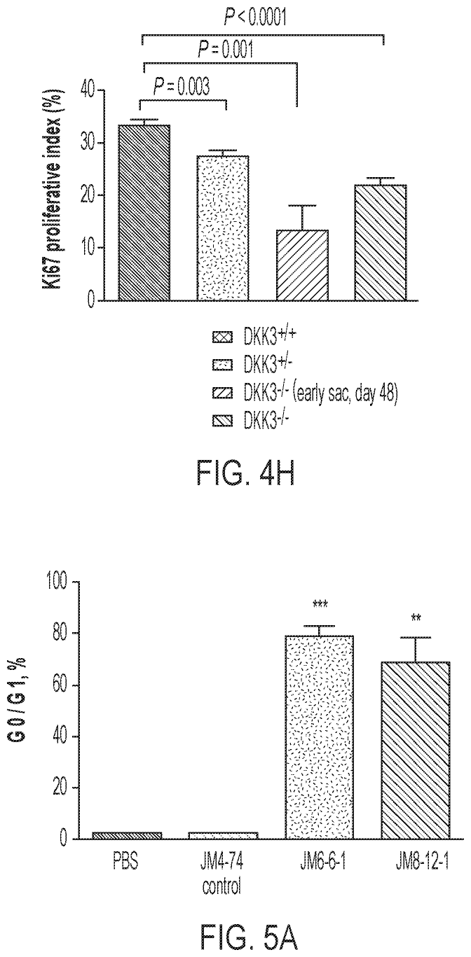

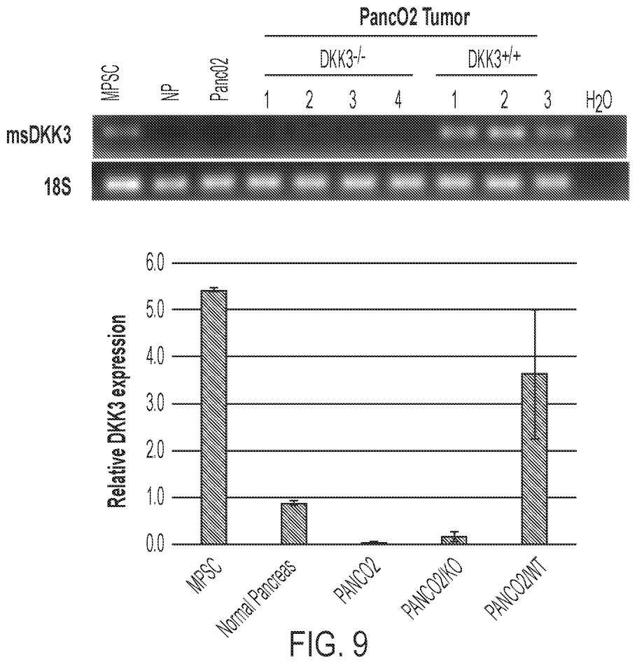

Using an orthotopic mouse model of PDAC in which HPSCs are co-injected with luciferase-labeled BxPC3 cells, the presence of HPSCs was found to stimulate increased growth of the primary tumor and distant metastases in a dose-dependent fashion (Hwang et al., 2008). To evaluate the role of DKK3 in tumor progression, nude mice were injected orthotopically with either BxPC3 cells alone or in combination with HPSC-shDKK3 or control cells (HPSC-shControl) at a tumor-to-stroma ratio of 1:0 or 1:3. Consistent with the previous observations, mice injected with both HPSCs and BxPC3 developed larger primary tumors with a higher rate of peritoneal metastases than did those injected with BxPC3 alone (FIG. 4A). However, co-implantation with HPSC-shDKK3 resulted in significantly smaller primary pancreatic tumors compared to co-implantation with HPSC-shControl with 67% reduction in tumor size (p<0.05, FIG. 4A) with a lower incidence of peritoneal metastases (25% vs 31%). In light of recent reports suggesting a role for DKK3 in immunomodulation, the effects of DKK3 were investigated in immune competent models of PDAC. A syngeneic implantation model was first used with luciferase-labeled murine pancreatic cancer cells Panc02 (negative for DKK3; FIG. 9) injected into either DKK3^{-/-} mice or control C57/BL6 mice. Tumor growth was exponential in control mice (FIG. 4B) but in DKK3^{-/-} mice, growth was significantly inhibited with 3.8-fold decrease in luciferase signal at 22 days (p<0.05) with far fewer Ki67-positive cells compared to controls (FIG. 4B). Together, these data support a stimulatory role of DKK3 in pancreatic tumor growth and metastasis.

### Example 6—Depletion of DKK3 Prolongs Survival in an Autochthonous Model of PDAC

To further investigate the effects of DKK3 on PDAC in an immune competent model, DKK3 was ablated in the KPC model of PDAC. DKK3-deficient mice on a C57/BL6 background (DKK3^{-/-} mice, gift from C. Niehrs, Mainz Germany) have been extensively characterized and have only minor physiologic changes and no evidence of cancer development at one year (Barrantes Idel et al., 2006). When bred with KPC mice, the resultant progeny, P48-Cre; Kras^{LSL-G12D};Trp53^{fl/fl}; dkk3^{-/-} (termed “KPC/DKK3^{-/-}), and their DKK3-heterozygous and DKK3-wild type littermates (KPC/DKK3+/− and KPC/DKK3^{+/+}) had normal phenotypes at birth. Mice were monitored until they were moribund and then euthanized, and survival was calculated. When DKK3 was depleted, either completely or partially (KPC/DKK3^{-/-} or KPC/DKK3^{+/-}), overall median survival was significantly prolonged compared to mice with wild-type DKK3 (68 days for KPC/DKK3^{-/-} and 63 days for KPC/DKK3+/− vs. 47 days for KPC/DKK3^{-/-}; p=0.0002; FIGS. 4C-D). Indeed, the mortality for mice with wild-type DKK3 was 5 times higher than that for mice with at least partial DKK3 depletion (HR 0.21, 95% CI 0.09-0.47 and HR 0.19, 95% CI 0.08-0.46; p=0.0002; FIG. 4D). A similar KPC model with heterozygous Trp53 has less aggressive disease and longer median survival (P48-Cre; Kras^{LSL-G12D};Trp53^{fl/+}; dkk3^{-/-}). When DKK3 was ablated in this slower-growing model, the difference in median survival between mice with intact or depleted DKK3 was even more striking with a greater than 2-fold increase in survival (83 days vs. 177 days, p<0.0001) and 25-fold difference in mortality (HR 0.04, p<0.0001; FIG. 10).

In all mice regardless of their DKK3 status, H&E staining confirmed the presence of pancreatic carcinoma at the time of euthanasia (FIG. 4E) despite the differences in survival with DKK3 ablation. As expected, DKK3 expression was virtually absent in homozygous KPC/DKK3^{-/-} mice (red bar) and at intermediate levels in heterozygous KPC/DKK3+/− mice (blue bar) compared to control KPC/DKK3^{+/+} (black bar) mice as measured by qPCR (FIG. 4F).

However, the activated stromal content was reduced with DKK3 ablation as shown by IHC analysis of α-SMA and collagen (FIGS. 4E and 4G) and the Ki67 proliferative index was significantly reduced in the tumors in KPC/DKK3^{-/-} mice compared with KPC/DKK3^{+/+} mice (22% vs. 33%, p<0.0001; FIGS. 4E-F). The reduction in Ki67 correlated in a dose-dependent manner with the degree of DKK3 ablation, from the highest expression in KPC/DKK3^{+/+} mice to the lowest in the KPC/DKK3^{-/-} mice. Taken together, these data indicate that pancreatic tumors eventually develop in all KPC mice regardless of the level of DKK3 expression, however when DKK3 expression is reduced, the tumors are less proliferative with less activated stroma.

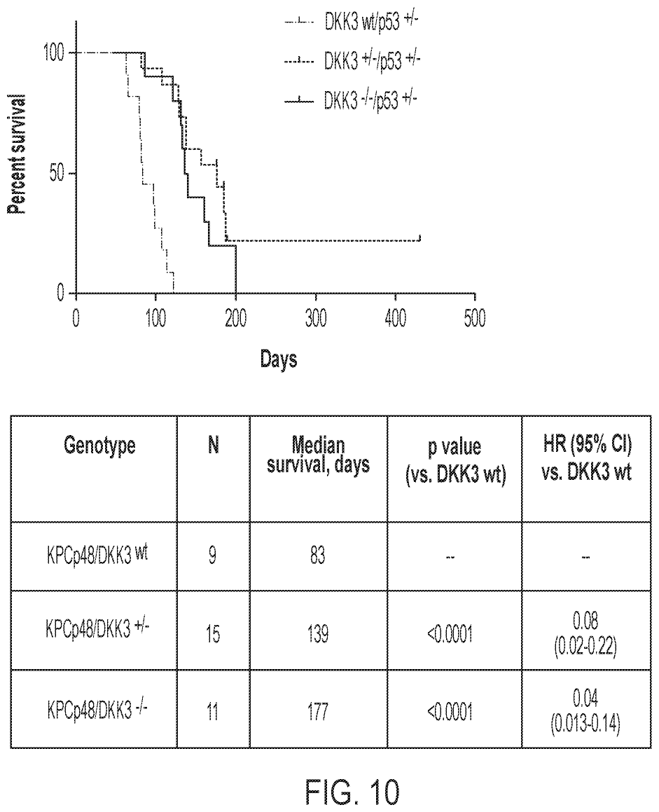

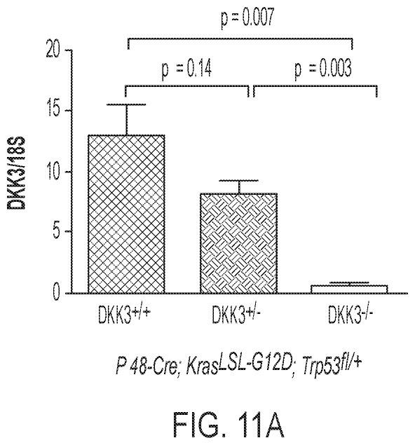

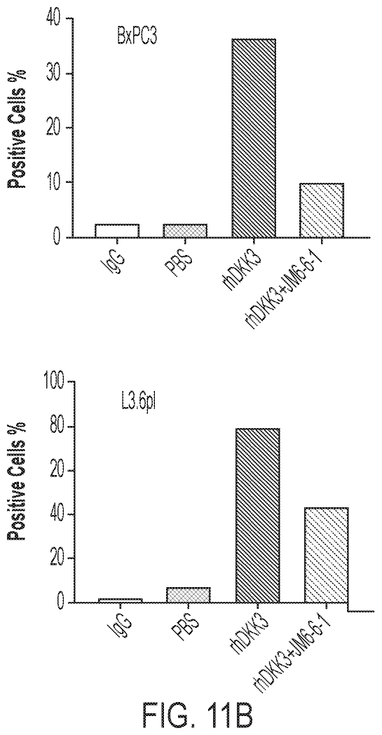

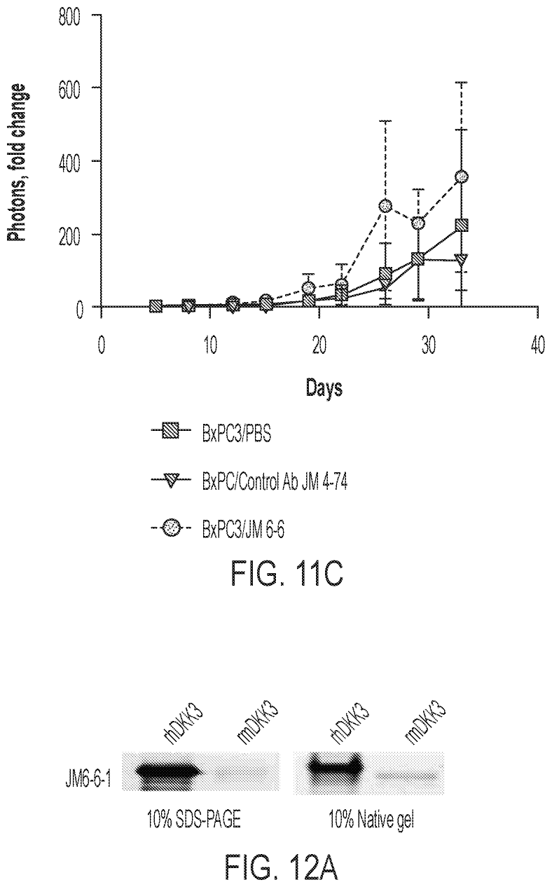

When the group of DKK3-heterozygous KPC mice was examined, survival was surprisingly similar to DKK3^{-/-} mice (FIGS. 4C-D; 63 vs. 68 days, p=NS) even though DKK3 expression by qPCR in KPC/DKK3+/− tumors was significantly higher (0=0.003). DKK3 expression in DKK3-heterozygous mice was 62% that of KPC/DKK3^{+/+} by qPCR (FIG. 4F). In the less aggressive P48-Cre; Kras^{LSL-G12D}; Trp53^{fl/+} model with heterozygous DKK3^{+/-} expression, DKK3 was similarly at 52.7% of DKK3^{+/+} mice (FIG. 11). These results suggest that even moderate depletion of DKK3 is effective in prolonging survival in this model of PDAC.

The extent to which DKK3 contributes to the earlier phases of PDAC development was then determined. KPC/DKK3^{-/-} mice were euthanized at about the same age (48 days) as when KPC/DKK3^{+/+} mice were dying (median survival, 47 days). At this timepoint, KPC/DKK3^{-/-} mice appeared healthy and H&E staining of their pancreas tissue was mostly normal with a few focal areas of dysplasia, but no foci of PanINs or invasive carcinoma (FIG. 4E) whereas the KPC/DKK3^{+/+} mice were moribund with pancreatic carcinoma. As expected, minimal activated stroma was seen on α-SMA staining, and the Ki67 proliferative index was significantly lower than that seen in the KPC/DKK3 wt mice at the same age (red hatched bar, p=0.001; FIGS. 4E-G). With time, however, activated stroma and cell proliferation increased as shown in the tumors collected when the mice were moribund (median survival, 68 days). These findings suggest that DKK3 may be involved in the early stages of pancreatic tumorigenesis and ablation of DKK3 delays the development of malignancy but does not completely prevent cancer formation.

When tumors do form in DKK3-depleted mice, the tumors are slow-growing with a low proliferative index and reduced activated stromal content, which may contribute to the improvement in overall survival. Histology and expression of α-SMA and Ki67 in DKK3-heterozygous tumors were similar to DKK3-null mice and are shown in FIGS. 4E and 4G.

### Example 7—Inhibition of DKK3 with a Neutralizing Antibody Suppresses PSCs and Cancer Cell Function, Enhances Response to Chemotherapy, and Prolongs Survival

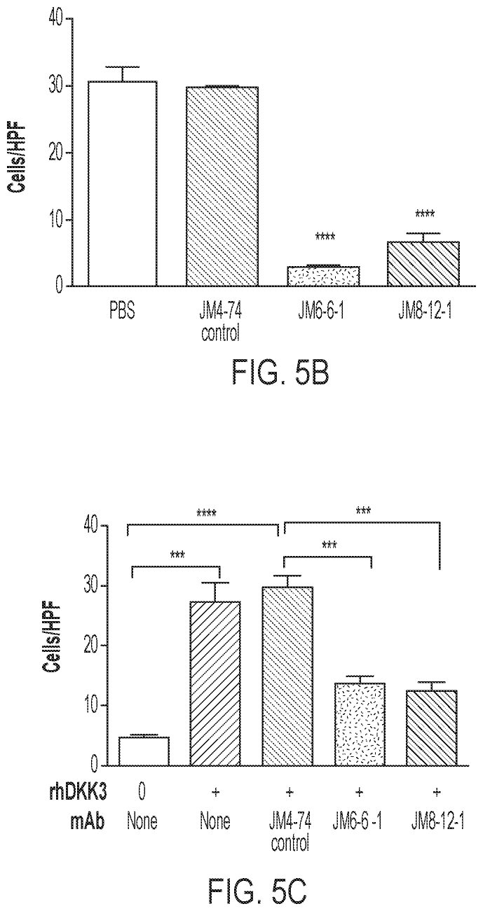

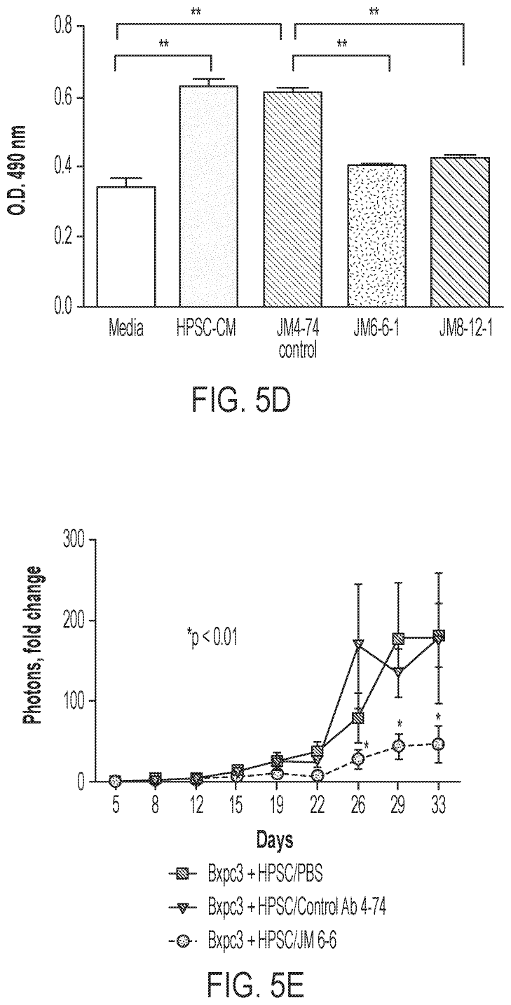

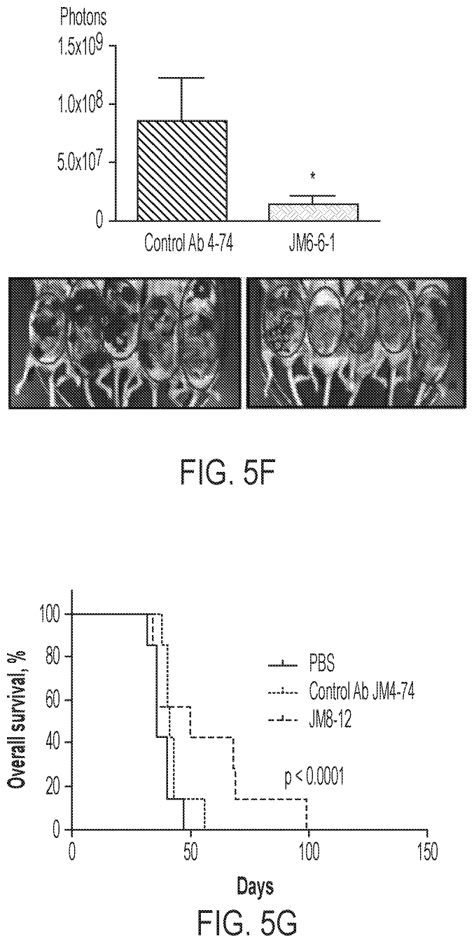

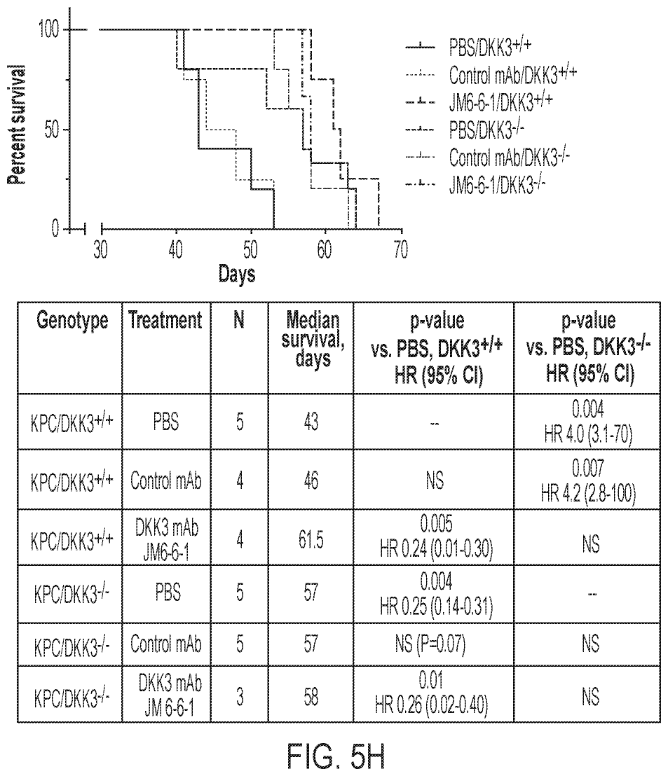

To further evaluate the therapeutic potential of DKK3 blockade, novel monoclonal antibodies (mAbs, clones JM6-6-1 and JM8-12-1; Tables 1-4) against human DKK3 were generated and their efficacy on HPSC and PDAC cell functions was tested. Treatment of HPSCs with either JM6-6-1 or JM8-12-1 induced growth arrest by 70- to 80-fold (p<0.01 and p<0.001; FIG. 5A) and inhibited migration by 5- to 11-fold (p<0.0001; FIG. 5B) compared with the irrelevant isotype control mAb. Treatment of BxPC3 cancer cells with JM6-6-1 or JM8-12-1 was able to reverse the DKK3-mediated induction of migration (p<0.001; FIG. 5C) and also abrogated the DKK3-mediated induction of chemoresistance to gemcitabine in cancer cells (FIG. 5D). In this assay, survival of BxPC3 cells in gemcitabine with HPSC-CM treatment was increased by 200% over controls but the addition of either DKK3 mAb clone restored sensitivity to gemcitabine with a proliferation rate similar to that of media controls (FIG. 5D). Cell surface binding of DKK3 on PDAC cells was assessed by flow cytometry which confirmed that addition of JM6-6-1 was effective in blocking binding of rhDKK3 to PDAC cells (FIG. 11B).

TABLE 1
CDRs of light chain variable sequences of
DKK3 antibodies

| Antibody | CDR1 | CDR2 | CDR3 |
|---|---|---|---|
| Name | (SEQ ID NO:) | (SEQ ID NO:) | (SEQ ID NO:) |
| JM8-12-2 | RASKSVSTSGYSYMH | LVSNLES | QHIRELTRS |
| | (SEQ ID NO: 1) | (SEQ ID | (SEQ ID |
| | | NO: 2) | NO: 3) |
| JM6-6-1 | RSSQSILHSNGHTYLE | KVSNRFS | FQGSHVPFT |
| | (SEQ ID NO: 4) | (SEQ ID | (SEQ ID |
| | | NO: 5) | NO: 6) |

TABLE 2
CDRs of heavy chain variable sequences of
DKK3 antibodies

| Antibody | CDR1 | CDR2 | CDR3 |
|---|---|---|---|
| Name | (SEQ ID NO:) | (SEQ ID NO:) | (SEQ ID NO:) |
| JM8-12-2 | GYSITSDYAWN | YISYRGSTR | DGYY |
| | (SEQ ID | (SEQ ID | (SEQ ID NO: 9) |
| | NO: 7) | NO: 8) | |
| JM6-6-1 | GFTFTIEYMA | SISSGGDDIY | SLSD |
| | (SEQ ID | (SEQ ID | (SEQ ID NO: 12) |
| | NO: 10) | NO: 11) | |

TABLE 3
Nucleotide sequences for antibody variable regions

| Antibody | | | SEQ |
|---|---|---|---|
| Name | Chain | Variable Sequence (5′ to 3′) | ID NO: |
| JM8-12-2 | Heavy | atgaaaccttctcagtctctgtccctcacctgcactgt | 13 |
| | | cactggctactcaatcaccagtgattatgcctggaact | |
| | | ggatccggcagtttccaggaaacaaactggagtggatg | |
| | | ggctacataagctacaggggtagtactaggtacaaccc | |
| | | atctctcaaaagtcgaatctctatcactcgagacacat | |
| | | ccaagaaccagctctacctgcagttgcattctgtgact | |
| | | actgaggacacagccacatattactgtgtccatgatgg | |
| | | ttactactgggtccaagggactctggtcactgtctctg | |
| | | cagccaaaacgacacccccatctgactat | |
| | Light | caggatccacgcgtagacattgtgctgacacagtctcc | 14 |
| | | tgcttccttagctgtatctctggggcagagggccacca | |
| | | tctcatacagggccagcaaaagtgtcagtacatctggc | |
| | | tatagttatatgcactggaaccaacagaaaccaggaca | |
| | | gccacccagactcctcatctatcttgtatccaacctag | |
| | | aatctggggtccctgccaggttcagtggcagtgggtct | |
| | | gggacagacttcaccctcaacatccatcctgtggagga | |
| | | ggaggatgctgcaacctattactgtcagcacattaggg | |
| | | agcttacacgttcggaggggggaccaag | |
| JM6-6-1 | Heavy | gaggtgaagctggtggagtctgggggaggcttagtgaa | 15 |
| | | gcctggagggtccctgaaactctcctgtgcagcctctg | |
| | | gattcactttcactatcgaatacatggcttggattcgc | |
| | | cagactcctgagaaaaggctggagtgggtcgcatccat | |
| | | tagtagtggtggtgatgacatctactatgcagacaatg | |
| | | tgaaggggcgattcaccatctccagagacaatgccaag | |
| | | aacaccctatacctgcaaatgagcagtctgaagtctga | |
| | | agacacagccatatattactgttcaagatctttatcgg | |
| | | actggggccaaggcaccactctcacggtctcctcagcc | |
| | | aaaacgacacccccatctgactatccactggcc | |
| | Light | atgacccaaactccactctccctgcctgtcagtcttgg | 16 |
| | | agatcaagcctccatctcttgcagatctagtcagagca | |
| | | ttttacatagtaatggacacacctatttagaatggtac | |
| | | ctgcagaaaccaggccagtctccaaagctcctgatcta | |
| | | caaagtttccaaccgattttctggggtcccagacaggt | |
| | | tcagtggcagtggatcagggacagatttcacactcaag | |
| | | atcagcagagtggaggctgaggatctgggagtttatta | |
| | | ctgctttcaaggttcacatgttccattcacgttcggct | |
| | | cgggaacaaagttggaaatagaacgggctgatgctgcc | |
| | | caa | |

TABLE 4
Protein sequences for antibody variable regions

| Antibody | | | SEQ ID |
|---|---|---|---|
| Name | Chain | Variable Sequence | NO: |
| JM8-12-2 | Heavy | MKPSQSLSLTCTVTGYSITSDYAWNWIRQFPGNKLEWM | 17 |
| | | GYISYRGSTRYNPSLKSRISITRDTSKNQLYLQLHSVT | |
| | | TEDTATYYCVHDGYYWVQQTLVTVSAAKTTPPSDY | |
| | Light | QDPRVDIVLTQSPASLAVSLFQRATISYRASKSVSTSG | 18 |
| | | YSYMHWNQQKPGQPPRLLIYLVSNLESGVPARFSGSGS | |
| | | GTDFTLNIHPVEEEDAATYYCQHIRELTRSEGGPSWK | |
| JM6-6-1 | Heavy | EVKLVESGGGLVKPGGSLKLSCAASGFTFTIEYMAWIR | 19 |
| | | QTPEKRLEWVASISSGGDDIYYADNVKGRFTISRDNAK | |
| | | NTLYLQMSSLKSEDTAIYYCSRSLSDWGQGTTLTVSSA | |
| | | KTTPPSDYPLA | |
| | Light | MTQTPLSLPVSLGDQASISCRSSQSILHSNGHTYLEWY | 20 |
| | | LQKPGQSPKLLIYKVSNRFSGVPDRFSGSGSGTDFTLK | |
| | | ISRVEAEDLGVYYCFQGSHVPFTFGSGTKLEIERADAA | |
| | | Q | |

The efficacy of DKK3 mAb was tested in vivo using the previously described orthotopic co-implantation nude mouse model of PDAC with human PSCs mixed with luciferase-labeled BxPC3 cancer cells (1:3 tumor:PSC ratio). Mice were treated with either phosphate-buffered saline (PBS), isotype control mAb, or DKK3 mAb (clone JM6-6-1; 5 mg/kg i.p. once every 5 days) and tumor growth was followed with IVIS imaging. Compared with either the PBS or control mAb groups, the JM6-6-1 mAb group showed a significant inhibition in tumor growth compared to baseline tumor signal at day 22 until day 33 when they were euthanized after completing 4 weeks of treatment (p<0.01; FIG. 5E). Tumors in the PBS and control mAb groups grew rapidly from day 22 onward, with an 8-fold increase in the luciferase signal. The tumors derived from BxPC3 alone without HPSCs showed no significant change in response to JM6-6-1 mAb treatment compared to either PBS or isotype control mAb (FIG. 11C), suggesting that JM6-6-1 is effective only when the target DKK3 is present and in this model, produced by the HPSCs. After pancreatic tumors were removed, examination of the peritoneal cavity also revealed a 6.5-fold lower volume of metastatic disease as measured by luciferase signal in the mice treated with JM6-6-1 compared with either PBS or the control mAb (p<0.05, FIG. 5F). In addition, treatment of the 4T1 mouse model of TNBC with DKK3 antibody JM6-6-1 improved survival (FIG. 13F).

In a separate survival experiment, treatment with JM8-12 (5 mg/kg i.p. once every 5 days) resulted in significant improvement in median survival compared to either control group (50 days for JM8-12, 36 days for PBS, 41 days for control mAb; p<0.0001; FIG. 5G). The hazard ratio for mice treated with JM8-12 mAb compared with that for the PBS control group was 0.26, indicating that in this xenograft orthotopic model of PDAC, treatment with anti-DKK3 mAb was associated with decreased tumor growth and metastasis with prolonged survival.

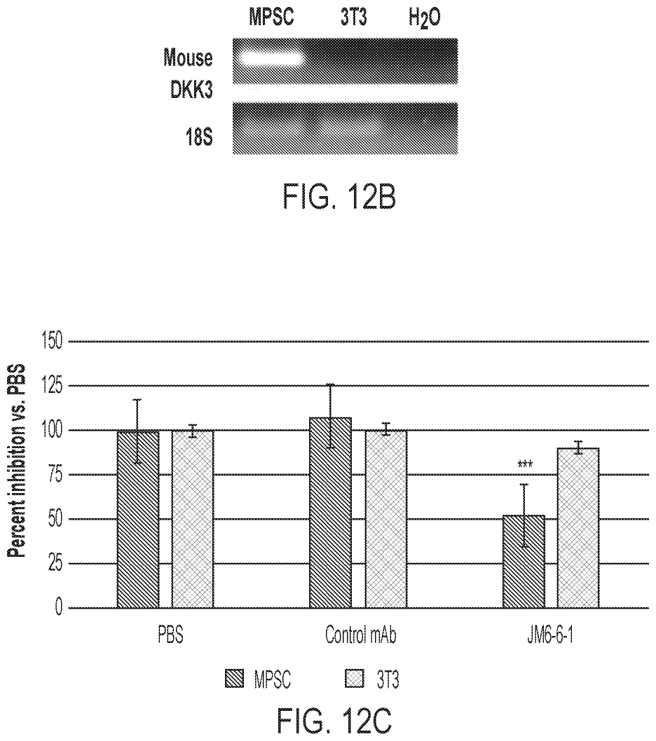

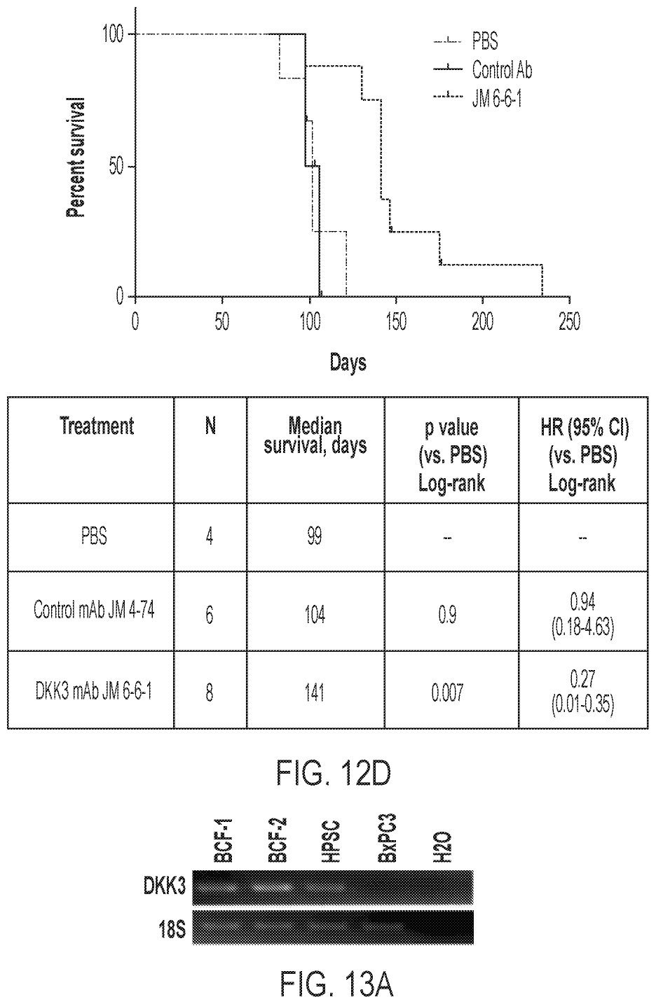

Since a significant improvement in survival was observed with genetic ablation of DKK3 in the KPC model of PDAC (FIG. 4), it sought to determine whether pharmacologic neutralization of DKK3 with mAb would also be effective in this model. Human and murine DKK3 share 83% protein sequence homology by BLAST analysis and therefore it was anticipated that it was likely that JM6-6-1 could cross-react with host DKK3. Cross-reactivity was confirmed by a non-denaturing Western blot, which showed that JM6-6-1 recognizes murine DKK3 although more weakly than human DKK3 (FIG. 12A). JM6-6-1 inhibited proliferation of PSCs isolated from mouse pancreas that express DKK3 (FIGS. 12B-C). When KPC mice (P48-Cre; Kras^{LSL-G12D};Trp53^{fl/fl}) were treated with JM6-6-1 (red solid line, FIG. 5H), median survival increased by 43% from 43 to 61.5 days compared to PBS or isotype control mAb (p=0.005, HR 0.24, 95% CI 0.01-0.30; FIG. 5H). In contrast, in KPC/DKK3^{-/-} mice that lack DKK3 (P48-Cre; Kras^{LSL-G12D}; Trp53^{fl/fl}; dkk3^{-/-}), JM6-6-1 had no effect on survival (red dashed line; 58 days) compared to PBS or control mAb treatment (black and blue dashed lines; 57 and 57 days respectively; p=NS) indicating that the effects of JM6-6-1 are likely to be specific for DKK3. Consistent with the previous results, genetic depletion of DKK3 in KPC mice was associated with improved survival (black dashed line; 57 days) compared to KPC/DKK3-wt mice treated with either PBS or isotype control mAb (black and blue solid lines; 43 and 46 days; p=0.004 and p=0.007). Interestingly, although the median survival of KPC/DKK3^{+/+} mice treated with JM6-6-1 was longer than that of KPC/DKK3^{-/-} mice (61.5 vs. 57 days), the difference was not statistically significant suggesting that pharmacologic neutralization of DKK3 using mAb was similar to genetic ablation to improve survival. JM6-6-1 was equally effective in another experiment with larger sample sizes using another well-accepted GEMM model of PDAC that uses the Pdx1 promoter to drive oncogenic Kras (Pdx1-Cre; Kras^{LSL-G12D}; Trp53^{fl/fl}) (FIG. 12D). However, this model was prone to development of benign papillomas, as previously reported (Lampson et al., 2012; Westphalen & Olive, 2012), and therefore subsequent studies were performed using the KPC model with the more pancreas-specific P48-Cre allele which did not develop papillomas.

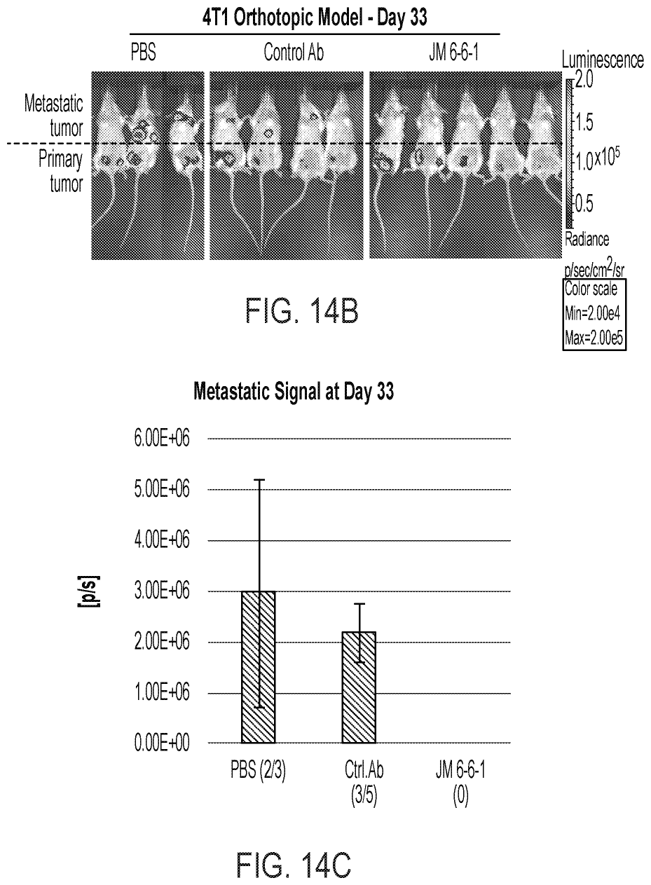

Treatment of TNBC orthotopic models with DKK3 mAb was also tested. An orthotopic model using 4T1 TNBC cells labeled with firefly luciferase was treated with PBS, a control antibody, or JM6-6-1. Tumor growth was measured 33 days after starting treatment and was compared to the initial tumor size prior to treatment. The tumors in the JM6-6-1-treated mice were significantly smaller compared to controls (FIG. 14A). The mice were also imaged to detect the primary tumors and any metastases (FIG. 14B). Quantification of the luciferase signal from 4T1 metastases shows that treatment with JM6-6-1 prevented metastases (FIG. 14C).

### Example 8—DKK3 Blockade is Associated with Increased Tumor Immune Infiltrates and Improves Response to Checkpoint Inhibitor Therapy

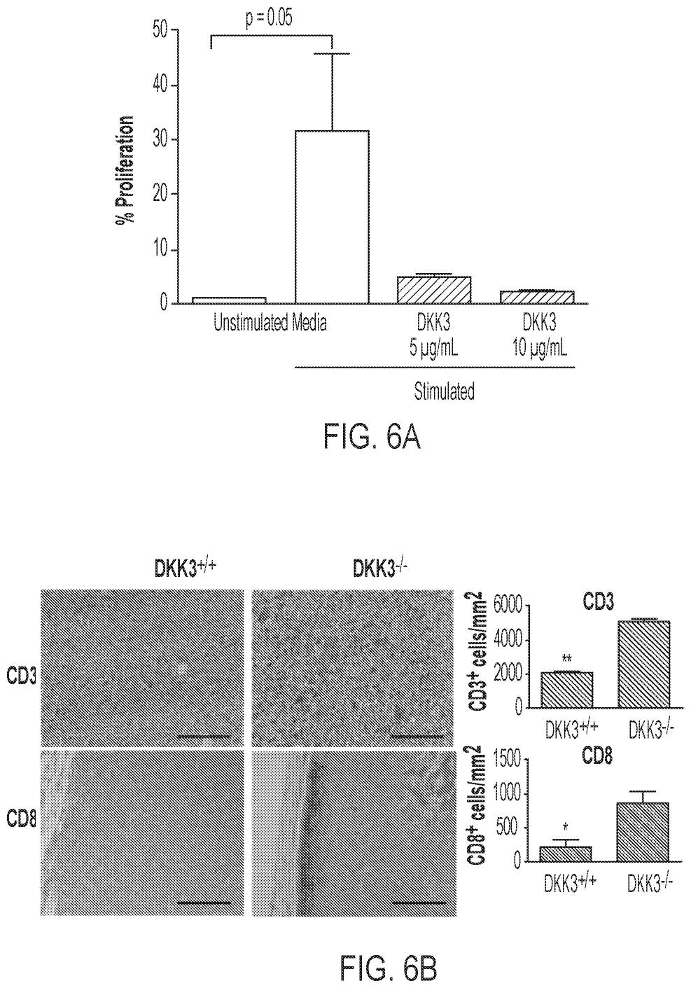

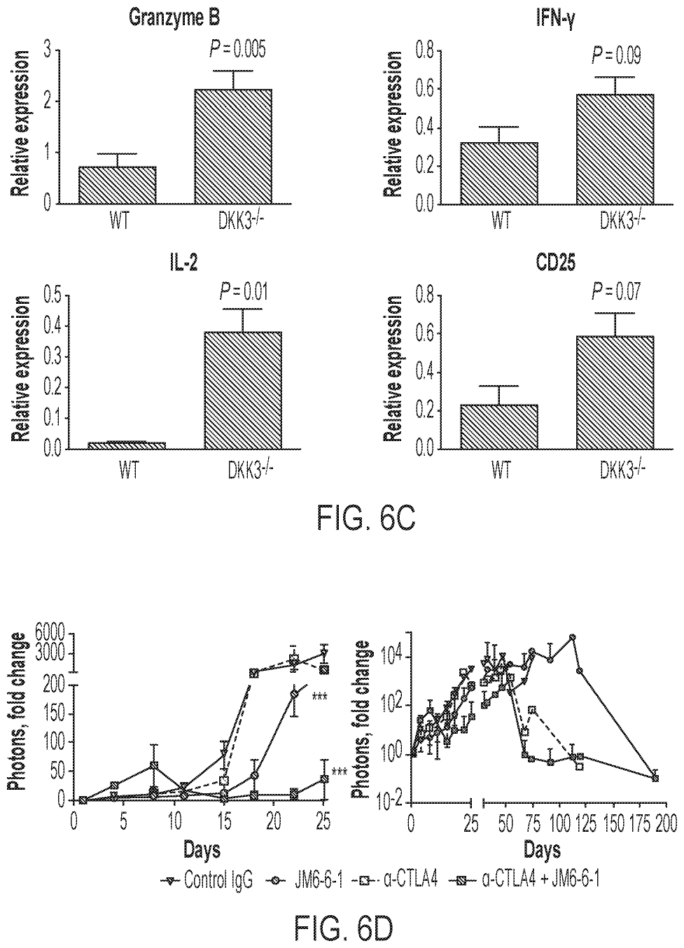

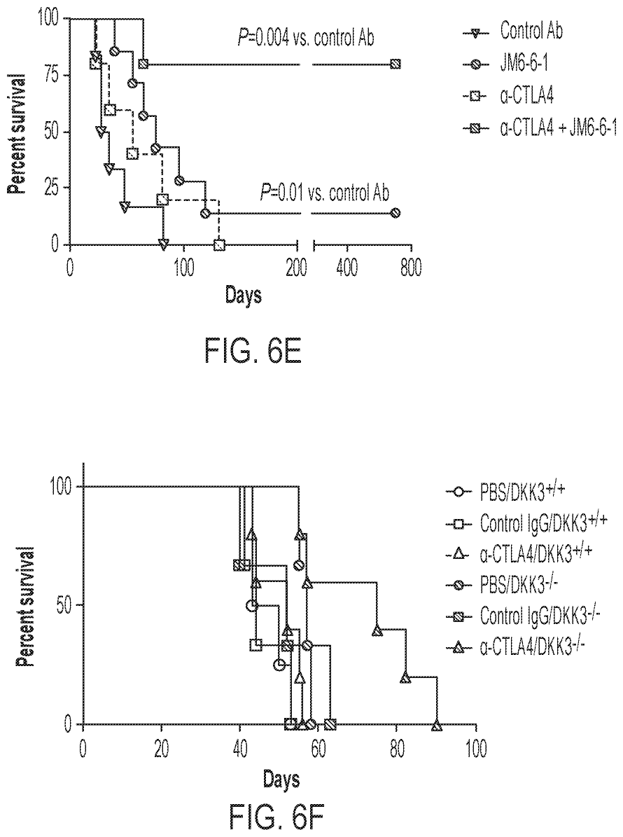

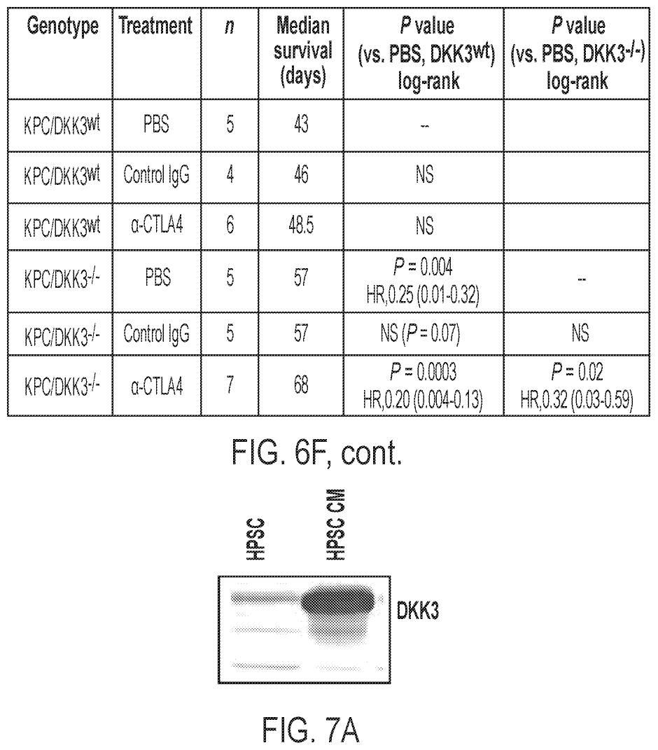

DKK3 has been shown to be an immune modulator and is associated with T-cell tolerance. When CD3+ T cells were stimulated with recombinant DKK3 in vitro, an inhibition of cell proliferation by 4.7-11.5 fold was observed compared to media alone (FIG. 6A; p<0.07). CD3 and CD8-expressing cells were analyzed in pancreatic tumors from a syngeneic orthotopic model with luciferase labeled KPC cells implanted in either DKK3^{-/-} or control C57/BL6 mice. IHC demonstrated a 2.4-fold increase in CD3+ cells in DKK3^{-/-} mice compared to controls (FIG. 6B). CD8+ cells were rarely seen in tumors from C57/BL6 mice but were consistently identified in the periphery of tumors from DKK3^{-/-} mice with a nearly 4-fold increase in expression (FIG. 6B). Additional markers of T cell activity were measured by qPCR which showed significant increases in granzyme B and IL-2 in DKK3^{-/-} tumors (p=0.005 and p=0.01, respectively) as well as increased IFN-gamma and CD25 although they were not-statistically significant (p=0.09 and p=0.07 respectively; FIG. 6C). These data suggest that DKK3 inhibited T cell proliferation and depletion of DKK3 was associated with increased CD3+ and CD8+ T cell numbers and activity in pancreatic tumors.

PDAC has been largely resistant to immune checkpoint therapies and several studies suggest this may be due to an immunosuppressive microenvironment (Johnson et al., 2017; Laheru & Jaffee, 2005; Zheng et al., 2013; Jiang & Hegde, 2016; Jiang et al., 2017; Kaneda et al., 2016; Jiang et al., 2016). In order to determine whether manipulation of DKK3 can affect the response to checkpoint inhibitors, mice in the syngeneic orthotopic C57/BL6 model were treated with either isotype control IgG, DKK3 mAb JM6-6-1, α-CTLA4 or the combination of JM6-6-1 with α-CTLA4. Treatment with α-CTLA4 alone was equivalent to control Ab treatment with no effect on tumor growth by luciferase signal (FIG. 6D). Treatment with JM6-6-1 alone resulted in growth inhibition at 22 days and onward compared to control IgG or α-CTLA4 (p<0.01). Mice in the combination group with JM6-6-1+α-CTLA4 showed inhibition of tumor growth after 8 days that was highly significant after day 18 (p<0.0001). With additional follow-up to day 200 (FIG. 6D, right), tumors in all groups continued to grow albeit at a slower rate in the JM6-6-1 and combination JM6-6-1+α-CTLA4 groups (FIG. 6D). Survival for the 4 treatment groups is shown in FIG. 6E. Compared to control IgG, median survival was significantly longer with JM6-6-1 alone (75 vs. 30 days, p=0.01) but was equivalent to α-CTLA4 treatment (p=0.32). Combination treatment JM6-6-1+α-CTLA4 was associated with a highly significant improvement in survival (p=0.004) and median survival was not reached in this group (FIG. 6E). Survival was also significantly better in the combination treatment group compared to JM6-6-1 alone (p=0.04). In the combination treatment group, 80% of mice were alive and appeared healthy at 800 days and were electively sacrificed. In the JM6-6-1 group, 20% of mice were still alive at 726 days when they were electively sacrificed. IVIS imaging was not performed after 200 days but at that point, there was minimal signal from the tumors in surviving mice (FIG. 6D) and no gross tumors were identified in the pancreata from the remaining mice in the JM6-6-1 and combination groups.

To further confirm these findings, the effect of α-CTLA4 was tested in the KPC/DKK3^{-/-} model of PDAC in a survival study (FIG. 6F). Control IgG and α-CTLA4 had no effect on survival compared to PBS in wild type KPC mice with intact DKK3 (black lines, median survival 43-48.5 days). As observed previously, median survival of KPC/DKK3^{-/-} mice was significantly improved compared to KPC/DKK3^{+/+} mice (57 vs. 43 days, p=0.004). When KPC/DKK3^{-/-} mice were treated with α-CTLA4, survival increased to 68 days, representing a 58% improvement compared to wild-type DKK3 with PBS treatment (p=0.0003; HR 0.20, 95% CI 0.004-0.13). The addition of α-CTLA4 to DKK3 ablation also improved survival on top of DKK3 ablation alone in KPC/DKK3^{-/-} mice (68 vs. 57 days; p=0.02; HR 0.32, 95% CI 0.03-0.59). In summary, depletion of DKK3 by either pharmacologic treatment with monoclonal Ab or genetic ablation improved PDAC response to checkpoint inhibitor immunotherapy with a significant improvement in survival.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Abarzua et al., Adenovirus-mediated overexpression of REIC/Dkk-3 selectively induces apoptosis in human prostate cancer cells through activation of c-Jun-NH2-kinase. Cancer Res., 65, 9617-9622 (2005).
Apte et al., Desmoplastic reaction in pancreatic cancer: role of pancreatic stellate cells. Pancreas, 29, 179-187 (2004).
Apte & Wilson, Dangerous liaisons: pancreatic stellate cells and pancreatic cancer cells. J. Gastroenterol. Hepatol., 27 Suppl 2, 69-74 (2012).
Arumugam et al., Effect of cromolyn on S100P interactions with RAGE and pancreatic cancer growth and invasion in mouse models. J. Natl. Cancer Inst., 98, 1806-1818 (2006).
Arumugam et al., Epithelial to mesenchymal transition contributes to drug resistance in pancreatic cancer. Cancer Res., 69, 5820-5828 (2009).
Barrantes Idel et al., Generation and characterization of dickkopf3 mutant mice. Molecular and Cellular Biology, 26, 2317-2326 (2006).
Beidler et al., J. Immunol., 141(11):4053-4060, 1988.
Bhowmick & Moses, Tumor-stroma interactions. Curr. Opin. Genet. Dev., 15, 97-101 (2005).
Bijlsma & van Laarhoven, The conflicting roles of tumor stroma in pancreatic cancer and their contribution to the failure of clinical trials: a systematic review and critical appraisal. Cancer Metastasis Rev., 34, 97-114 (2015).
Caricasole et al., Functional characterization of WNT7A signaling in PC12 cells: interaction with A FZDS×LRP6 receptor complex and modulation by Dickkopf proteins. J. Biol. Chem., 278, 37024-37031 (2003).
Cowling, C. M. D'Cruz, L. A. Chodosh, M. D. Cole, c-Myc transforms human mammary epithelial cells through repression of the Wnt inhibitors DKK1 and SFRP1. Mol. Cell. Biol. 27, 5135-5146 (2007).
Dvorak, Tumors: wounds that do not heal. Similarities between tumor stroma generation and wound healing. N. Engl. J. Med., 315, 1650-1659 (1986).
Edamura et al., Adenovirus-mediated REIC/Dkk-3 gene transfer inhibits tumor growth and metastasis in an orthotopic prostate cancer model. Cancer Gene Ther., 14, 765-772 (2007).
Frazier et al., Establishment of a new human pancreatic adenocarcinoma cell line, MDAPanc-3. Pancreas, 5, 8-16 (1990).
Guder et al., An ancient Wnt-Dickkopf antagonism in Hydra. Development, 133, 901-911 (2006).
Hingorani et al., Preinvasive and invasive ductal pancreatic cancer and its early detection in the mouse. Cancer Cell, 4, 437-450 (2003).
Hingorani et al., Trp53R172H and KrasG12D cooperate to promote chromosomal instability and widely metastatic pancreatic ductal adenocarcinoma in mice. Cancer Cell, 7, 469-483 (2005).
Hoang et al., Dickkopf 3 Inhibits Invasion and Motility of Saos-2 Osteosarcoma Cells by Modulating the Wnt-{beta}-Catenin Pathway. Cancer Res., 64, 2734-2739 (2004).
Hsieh et al., Dickkopf-3//REIC functions as a suppressor gene of tumor growth. Oncogene, 23, 9183-9189 (2004).
Hwang et al., Cancer-Associated Stromal Fibroblasts Promote Pancreatic Tumor Progression. Cancer Res., 68, 918-926 (2008).
Hwang et al., Cancer-associated stromal fibroblasts promote pancreatic tumor progression. Cancer Res, 68, 918-926 (2008).
Ji et al., Ras Activity Levels Control the Development of Pancreatic Diseases. Gastroenterology, 137, 1072-1082 (2009).
Jiang et al., Targeting focal adhesion kinase renders pancreatic cancers responsive to checkpoint immunotherapy. Nat. Med., 22, 851-860 (2016).
Jiang et al., Tumor-associated fibrosis as a regulator of tumor immunity and response to immunotherapy. Cancer Immunol. Immunother., 66, 1037-1048 (2017).
Johnson et al., Strategies for Increasing Pancreatic Tumor Immunogenicity. Clin. Cancer Res., 23, 1656-1669 (2017).
Jones et al., Nature, 321:522-525, 1986.
Kalluri & Zeisberg, Fibroblasts in cancer. Nat. Rev. Cancer, 6, 392-401 (2006).
Kaneda et al., Macrophage PI3Kgamma Drives Pancreatic Ductal Adenocarcinoma Progression. Cancer Discov., 6, 870-885 (2016).
Katase et al., Absence of Dickkopf (Dkk)-3 protein expression is correlated with longer disease-free survival and lower incidence of metastasis in head and neck squamous cell carcinoma. Oncol. Lett., 3, 273-280 (2012).
Kawasaki et al., REIC/Dkk-3 overexpression downregulates P-glycoprotein in multidrug-resistant MCF7/ADR cells and induces apoptosis in breast cancer. Cancer Gene Ther.,
16, 65-72 (2009).
Kuphal et al., Expression of Dickkopf genes is strongly reduced in malignant melanoma. Oncogene, 25, 5027-5036 (2006).
Laheru & Jaffee, Immunotherapy for pancreatic cancer—science driving clinical progress. Nat. Rev. Cancer, 5, 459-467 (2005).
Lampson et al., Targeting eNOS in pancreatic cancer. Cancer Res., 72, 4472-4482 (2012).
Li et al., Overexpression of oncogenic STK15/BTAK/Aurora A kinase in human pancreatic cancer. Clin. Cancer Res., 9, 991-997 (2003).
Logsdon et al., Molecular profiling of pancreatic adenocarcinoma and chronic pancreatitis identifies multiple genes differentially regulated in pancreatic cancer. Cancer Res., 63, 2649-2657 (2003).
Lu et al., Dickkopf-3 Contributes to the Regulation of Anti-Tumor Immune Responses by
Mesenchymal Stem Cells. Front. Immunol., 6, 645 (2015).
Macheda & Stacker, Importance of Wnt signaling in the tumor stroma microenvironment. Curr. Cancer Drug Targets, 8, 454-465 (2008).
Meister et al., Dickkopf-3, a tissue-derived modulator of local T-cell responses. Front. Immunol., 6, 78 (2015).
Moon et al., WNT and beta-catenin signalling: diseases and therapies. Nat. Rev. Genet., 5, 691-701 (2004).
Mueller & Fusenig, Friends or foes-bipolar effects of the tumour stroma in cancer. Nature Reviews Cancer, 4, 839-849 (2004).
Nakamura et al., Identification of two novel activities of the Wnt signaling regulator Dickkopf 3 and characterization of its expression in the mouse retina. BMC Cell Biol., 8, 52 (2007).
Nakamura & Hackam, Analysis of Dickkopf3 interactions with Wnt signaling receptors. Growth Factors, 28, 232-242 (2010).
Niehrs, Function and biological roles of the Dickkopf family of Wnt modulators. Oncogene, 25, 7469-7481 (2006).
Niu et al., Keratinocyte growth factor/fibroblast growth factor-7-regulated cell migration and invasion through activation of NF-kappaB transcription factors. J. Biol. Chem., 282, 6001-6011 (2007).
Nozaki et al., Reduced expression of REIC/Dkk-3 gene in non-small cell lung cancer. Int. J. Oncol., 19, 117-121 (2001).
Olive et al., Inhibition of Hedgehog signaling enhances delivery of chemotherapy in a mouse model of pancreatic cancer. Science, 324, 1457-1461 (2009).
Padua, Massague, Roles of TGF[beta] in metastasis. Cell Res., 19, 89-102 (2009).
Papatriantafyllou et al., Dickkopf-3, an immune modulator in peripheral CD8 T-cell tolerance. Proc. Natl. Acad. Sci. U.S.A., 109, 1631-1636 (2012).
Sakaguchi et al., Overexpression of REIC/Dkk-3 in Normal Fibroblasts Suppresses Tumor Growth via Induction of Interleukin-7. J. Biol. Chem., 284, 14236-14244 (2009).
Shaw et al., J. Natl. Cancer Inst., 80(19):1553-1559, 1988.
Shou et al., Human Dkk-1, a gene encoding a Wnt antagonist, responds to DNA damage and its overexpression sensitizes brain tumor cells to apoptosis following alkylation damage of DNA. Oncogene, 21, 878-889 (2002).
Sun et al., J. Steroid Biochem., 26(1):83-92, 1987.
Suzawa et al., Distant Bystander Effect of REIC/DKK3 Gene Therapy Through Immune System Stimulation in Thoracic Malignancies. Anticancer Res., 37, 301-307 (2017).
Taipale & Beachy, The Hedgehog and Wnt signalling pathways in cancer. Nature, 411, 349-354 (2001).
Tanimoto et al., REIC/Dkk-3 as a potential gene therapeutic agent against human testicular cancer. Int. J. Mol. Med., 19, 363-368 (2007).
Than et al., Intraperitoneal administration of an adenovirus vector carrying REIC/Dkk-3 suppresses peritoneal dissemination of scirrhous gastric carcinoma. Oncol. Rep., 25, 989-995 (2011).
Uchida et al., Potential of adenovirus-mediated REIC/Dkk-3 gene therapy for use in the treatment of pancreatic cancer. J. Gastroenterol. Hepatol., 29, 973-983 (2014).
Uchida et al., Synergistic anti-pancreatic cancer immunological effects by treatment with REIC/DKK3 protein and peripheral blood mononuclear cells. J. Gastroenterol. Hepatol., (2015).
Uchida et al., Synergistic anti-pancreatic cancer immunological effects by treatment with reduced expression in immortalized cells/dickkopf-3 protein and peripheral blood mononuclear cells. J. Gastroenterol. Hepatol., 31, 1154-1159 (2016).
Westphalen & Olive, Genetically engineered mouse models of pancreatic cancer. Cancer J., 18, 502-510 (2012).
Wood et al., J. Clin. Lab. Immunol., 17(4):167-171, 1985.
Wu et al., Mutual antagonism between dickkopf1 and dickkopf2 regulates Wnt/beta-catenin signalling. Curr. Biol., 10, 1611-1614 (2000).
Xu et al., Role of pancreatic stellate cells in pancreatic cancer metastasis. Am. J. Pathol., 177, 2585-2596 (2010).
Yamada et al., Establishment of a human pancreatic adenocarcinoma cell line (PSN-1) with amplifications of both c-myc and activated c-Ki-ras by a point mutation. Biochem. Biophys. Res. Commun., 140, 167-173 (1986).
Yue et al., Downregulation of Dkk3 activates beta-catenin/TCF-4 signaling in lung cancer. Carcinogenesis, 29, 84-92 (2008).
Zenzmaier et al., Dickkopf-related protein 3 promotes pathogenic stromal remodeling in benign prostatic hyperplasia and prostate cancer. Prostate, 73, 1441-1452 (2013).
Zheng et al., Role of immune cells and immune-based therapies in pancreatitis and pancreatic ductal adenocarcinoma. Gastroenterology, 144, 1230-1240 (2013).

1. A monoclonal antibody or antibody fragment, wherein the antibody or antibody fragment comprises:
a heavy chain variable region (VH) comprising a VHCDR1 amino acid sequence of SEQ ID NO: 7, a VHCDR2 amino acid sequence of SEQ ID NO: 8, and a VHCDR3 amino acid sequence of SEQ ID NO: 9; and a light chain variable region (VL) comprising a VLCDR1 amino acid sequence of SEQ ID NO: 1, a VLCDR2 amino acid sequence of SEQ ID NO: 2, and a VLCDR3 amino acid sequence of SEQ ID NO: 3; or
a heavy chain variable region (VH) comprising a VHCDR1 amino acid sequence of SEQ ID NO: 10, a VHCDR2 amino acid sequence of SEQ ID NO: 11, and a VHCDR3 amino acid sequence of SEQ ID NO: 12; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6.

2. The monoclonal antibody or antibody fragment of claim 1, wherein said antibody or antibody fragment is encoded by a heavy chain variable sequence having at least 70%, 80%, or 90% identity to SEQ ID NO: 13 and a light chain variable sequence having at least 70%, 80%, or 90% identity to SEQ ID NO: 14.

3. The monoclonal antibody or antibody fragment of claim 1, wherein said antibody or antibody fragment is encoded by a heavy chain variable sequence having at least 70%, 80%, or 90% identity to SEQ ID NO: 15 and a light chain variable sequence having at least 70%, 80%, or 90% identity to SEQ ID NO: 16.

4. The monoclonal antibody or antibody fragment of claim 1, wherein said antibody or antibody fragment is encoded by a heavy chain variable sequence having at least 95% identity to SEQ ID NO: 13 and a light chain variable sequence having at least 95% identity to SEQ ID NO: 14.

5. The monoclonal antibody or antibody fragment of claim 1, wherein said antibody or antibody fragment is encoded by a heavy chain variable sequence having at least 95% identity to SEQ ID NO: 15 and a light chain variable sequence having at least 95% identity to SEQ ID NO: 16.

6. The monoclonal antibody or antibody fragment of claim 1, wherein said antibody or antibody fragment is encoded by a heavy chain variable sequence according to SEQ ID NO: 13 and a light chain variable sequence according to SEQ ID NO: 14.

7. The monoclonal antibody or antibody fragment of claim 1, wherein said antibody or antibody fragment is encoded by a heavy chain variable sequence according to SEQ ID NO: 15 and a light chain variable sequence according to SEQ ID NO: 16.

8. The monoclonal antibody or antibody fragment of claim 1, wherein said antibody or antibody fragment comprises a heavy chain variable sequence having at least 70%, 80%, or 90% identity to SEQ ID NO: 17 and a light chain variable sequence having at least 70%, 80%, or 90% identity to SEQ ID NO: 18.

9. The monoclonal antibody or antibody fragment of claim 1, wherein said antibody or antibody fragment comprises a heavy chain variable sequence having at least 70%, 80%, or 90% identity to SEQ ID NO: 19 and a light chain variable sequence having at least 70%, 80%, or 90% identity to SEQ ID NO: 20.

10. The monoclonal antibody or antibody fragment of claim 1, wherein said antibody or antibody fragment comprises a heavy chain variable sequence having at least 95% identity to SEQ ID NO: 17 and a light chain variable sequence having at least 95% identity to SEQ ID NO: 18.

11. The monoclonal antibody or antibody fragment of claim 1, wherein said antibody or antibody fragment comprises a heavy chain variable sequence having at least 95% identity to SEQ ID NO: 19 and a light chain variable sequence having at least 95% identity to SEQ ID NO: 20.

12. The monoclonal antibody or antibody fragment of claim 1, wherein said antibody or antibody fragment comprises a heavy chain variable sequence having a sequence according to SEQ ID NO: 17 and a light chain variable sequence having a sequence according to SEQ ID NO: 18.

13. The monoclonal antibody or antibody fragment of claim 1, wherein said antibody or antibody fragment comprises a heavy chain variable sequence having a sequence according to SEQ ID NO: 19 and a light chain variable sequence having a sequence according to SEQ ID NO: 20.

14. The monoclonal antibody or antibody fragment of claim 1, wherein said antibody or antibody fragment is a humanized antibody.

15. The monoclonal antibody or antibody fragment of any one of claims 1-14, wherein the antibody fragment is a monovalent scFv (single chain fragment variable) antibody, divalent scFv, Fab fragment, F(ab′)₂ fragment, F(ab′)₃ fragment, Fv fragment, or single chain antibody.

16. The monoclonal antibody or antibody fragment of any one of claims 1-14, wherein said antibody is a chimeric antibody or bispecific antibody.

17. The monoclonal antibody or antibody fragment of any one of claims 1-16, wherein said antibody is an IgG antibody or a recombinant IgG antibody or antibody fragment.

18. The monoclonal antibody or antibody fragment of any one of claims 1-17, wherein the antibody is conjugated or fused to an imaging agent or a cytotoxic agent.

19. A monoclonal antibody or an antigen binding fragment thereof, which competes for binding to the same epitope as the monoclonal antibody or an antigen-binding fragment thereof according to any one of claims 1-17.

20. The monoclonal antibody or antibody fragment of claim 19, wherein said antibody or antibody fragment is a humanized antibody.

21. The monoclonal antibody or antibody fragment of claim 19 or 20, wherein the antibody fragment is a monovalent scFv (single chain fragment variable) antibody, divalent scFv, Fab fragment, F(ab′)₂ fragment, F(ab′)₃ fragment, Fv fragment, or single chain antibody.

22. The monoclonal antibody or antibody fragment of any one of claims 19-21, wherein said antibody is a chimeric antibody or bispecific antibody.

23. The monoclonal antibody or antibody fragment of any one of claims 19-22, wherein said antibody is an IgG antibody or a recombinant IgG antibody or antibody fragment.

24. The monoclonal antibody or antibody fragment of any one of claims 19-23, wherein the antibody is conjugated or fused to an imaging agent or a cytotoxic agent.

25. A hybridoma or engineered cell encoding an antibody or antibody fragment of any one of claim 1-17 or 19-23.

26. A method of treating a patient having a cancer, the method comprising administering an effective amount of a DKK3-neutralizing antibody or antibody fragment.

27. The method of claim 26, wherein the DKK3-neutralizing antibody or antibody fragment is the antibody or antibody fragment of any one of claims 1-24.

28. The method of claim 26, further defined as a method for increasing sensitivity to chemotherapy.

29. The method of claim 26, further defined as a method for increasing sensitivity to immunotherapy.

30. The method of claim 26, wherein the cancer is a pancreatic cancer, breast cancer, ovarian cancer, gastric cancer, bladder cancer, or sarcoma.

31. The method of claim 30, wherein the breast cancer is triple-negative breast cancer.

32. The method of claim 30, further defined as a method of inhibiting cancer metastasis.

33. The method of claim 30, further defined as a method of inhibiting cancer growth.

34. The method of claim 26, further comprising administering at least a second anti-cancer therapy.

35. The method of claim 34, wherein the second anti-cancer therapy is a chemotherapy, immunotherapy, radiotherapy, gene therapy, surgery, hormonal therapy, anti-angiogenic therapy or cytokine therapy.

36. The method of claim 35, wherein the chemotherapy comprises gemcitabine.

37. The method of claim 35, wherein the immunotherapy comprises an immune checkpoint inhibitor.

38. The method of claim 37, wherein the immune checkpoint inhibitor is a CTLA-4 antagonist, a PD-1 antagonist, a PD-L1 antagonist, an OX40 agonist, a LAGS antagonist, a 4-1BB agonist, or a TIM3 antagonist.

39. The method of claim 38, wherein the immune checkpoint inhibitor is a combination of a CTLA-4 antagonist and a PD1 antagonist.

40. The method of claim 38, wherein the immune checkpoint inhibitor is a combination of a CTLA-4 antagonist and a PDL1 antagonist.


SEQUENCE LISTING (20 sequences)

SEQ ID NO: 1 - protein, 15 aa, Artificial Sequence
  Synthetic polypeptide
RASKSVSTSGYSYMH

SEQ ID NO: 2 - protein, 7 aa, Artificial Sequence
  Synthetic polypeptide
LVSNLES

SEQ ID NO: 3 - protein, 9 aa, Artificial Sequence
  Synthetic polypeptide
QHIRELTRS

SEQ ID NO: 4 - protein, 16 aa, Artificial Sequence
  Synthetic polypeptide
RSSQSILHSNGHTYLE

SEQ ID NO: 5 - protein, 7 aa, Artificial Sequence
  Synthetic polypeptide
KVSNRFS

SEQ ID NO: 6 - protein, 9 aa, Artificial Sequence
  Synthetic polypeptide
FQGSHVPFT

SEQ ID NO: 7 - protein, 11 aa, Artificial Sequence
  Synthetic polypeptide
GYSITSDYAWN

SEQ ID NO: 8 - protein, 9 aa, Artificial Sequence
  Synthetic polypeptide
YISYRGSTR

SEQ ID NO: 9 - protein, 4 aa, Artificial Sequence
  Synthetic polypeptide
DGYY

SEQ ID NO: 10 - protein, 10 aa, Artificial Sequence
  Synthetic polypeptide
GFTFTIEYMA

SEQ ID NO: 11 - protein, 10 aa, Artificial Sequence
  Synthetic polypeptide
SISSGGDDIY

SEQ ID NO: 12 - protein, 4 aa, Artificial Sequence
  Synthetic polypeptide
SLSD

SEQ ID NO: 13 - DNA, 333 bp, Artificial Sequence
  Synthetic polynucleotide
atgaaaccttctcagtctctgtccctcacctgcactgtcactggctactcaatcaccagtgattatgcctggaactggatccggcagtttccaggaaacaaactggagtggatgggctacataagctacaggggtagtactaggtacaacccatctctcaaaagtcgaatctctatcactcgagacacatccaagaaccagctctacctgcagttgcattctgtgactactgaggacacagccacatattactgtgtccatgatggttactactgggtccaagggactctggtcactgtctctgcagccaaaacgacacccccatctgactat

SEQ ID NO: 14 - DNA, 332 bp, Artificial Sequence
  Synthetic polynucleotide
caggatccacgcgtagacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaag

SEQ ID NO: 15 - DNA, 375 bp, Artificial Sequence
  Synthetic polynucleotide
gaggtgaagctggtggagtctgggggaggcttagtgaagcctggagggtccctgaaactctcctgtgcagcctctggattcactttcactatcgaatacatggcttggattcgccagactcctgagaaaaggctggagtgggtcgcatccattagtagtggtggtgatgacatctactatgcagacaatgtgaaggggcgattcaccatctccagagacaatgccaagaacaccctatacctgcaaatgagcagtctgaagtctgaagacacagccatatattactgttcaagatctttatcggactggggccaaggcaccactctcacggtctcctcagccaaaacgacacccccatctgactatccactggcc

SEQ ID NO: 16 - DNA, 345 bp, Artificial Sequence
  Synthetic polynucleotide
atgacccaaactccactctccctgcctgtcagtcttggagatcaagcctccatctcttgcagatctagtcagagcattttacatagtaatggacacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcgggaacaaagttggaaatagaacgggctgatgctgcccaa

SEQ ID NO: 17 - protein, 111 aa, Artificial Sequence
  Synthetic polypeptide
MKPSQSLSLTCTVTGYSITSDYAWNWIRQFPGNKLEWMGYISYRGSTRYNPSLKSRISITRDTSKNQLYLQLHSVTTEDTATYYCVHDGYYWVQQTLVTVSAAKTTPPSDY

SEQ ID NO: 18 - protein, 113 aa, Artificial Sequence
  Synthetic polypeptide
QDPRVDIVLTQSPASLAVSLFQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 19 - protein, 125 aa, Artificial Sequence
  Synthetic polypeptide
EVKLVESGGGLVKPGGSLKLSCAASGFTFTIEYMAWIRQTPEKRLEWVASISSGGDDIYYADNVKGRFTISRDNAKNTLYLQMSSLKSEDTAIYYCSRSLSDWGQGTTLTVSSAKTTPPSDYPLA

SEQ ID NO: 20 - protein, 115 aa, Artificial Sequence
  Synthetic polypeptide
MTQTPLSLPVSLGDQASISCRSSQSILHSNGHTYLEWYLQKPGQSPKLLIYKVSNRFSGVPDRFSGSGSGTDFTLKISRVEAEDLGVYYCFQGSHVPFTFGSGTKLEIERADAAQ